(19) United States
(12) Patent Application Publication
(10) Pub. No.: US 2022/0145036 A1
(43) Pub. Date: May 12, 2022

(54) THERMOPLASTIC FOAM PREPARED FROM TWO SPECIAL POLYURETHANE DISPERSIONS

(71) Applicant: Covestro Intellectual Property GmbH & Co. KG, Leverkusen (DE)

(72) Inventors: Marc-Stephan Weiser, Kürten-Dürscheid (DE); Sascha Plug, Leverkusen (DE); Sebastian Doerr, Düsseldorf (DE)

(21) Appl. No.: 17/429,398

(22) PCT Filed: Feb. 18, 2020
(86) PCT No.: PCT/EP2020/054228

§ 371 Date: Aug. 9, 2021

(30) Foreign Application Priority Data
Feb. 28, 2019 (EP) 19159939.8

(51) Int. Cl. C08J 9/28; C08G 18/12; C08G 18/73; C08G 18/75; C08G 18/72; C08G 18/48; A61L 15/42; A61L 15/26

(52) U.S. Cl. CPC C08J 9/286; C08G 18/12; C08G 18/73; C08G 18/755; C08G 18/722; C08J 2201/0504; C08G 18/4854; A61L 15/425; A61L 15/26; C08J 2375/08; C08J 2207/12; C08G 18/4829

(57) ABSTRACT

The invention relates to a foamed, fissure-free skin-compatible article, produced by mixing a first polyurethane dispersion (A) with at least one second polyurethane dispersion (B) which differs from the first polyurethane dispersion (A), optionally with the addition of other additives, then by foaming and subsequently drying the mixture. The invention also relates to a process for preparing the foamed article and to the use thereof.

The invention relates to a foamed article, especially a hygiene article, a constituent of a wound dressing, a constituent of a wearable device or a wound-dressing foam, obtainable on the basis of two polyurethane dispersions (A) and (B), and to the production process therefor, the use thereof and the provision of a kit of parts from the two polyurethane dispersions (A) and (B).

The prior art discloses foamed articles that require various foam stabilizers for provision of a stable foam. The foam stabilizers used are usually unsuitable for human skin contact or for production of wound dressings therefrom. This is particularly because of the cytotoxic potential of the additives used in the formulations used. Such foams are known from patent application US-2016-0235880-A1 or US-2011-0275728-A1.

It is frequently a further demand on foamed articles that they should be very substantially crack-free. This is particularly important in turn when these foamed articles are to find use as wound dressing. The cracks have the disadvantage both that they impair the stability of the foamed article overall, but are also traps for impurities and usually suggest a certain brittleness of the foam, and are visually unacceptable to consumers.

Therefore, it was an object of the present invention to provide a foamed article or a production process for such an article that at least partly does not have at least one disadvantage of the prior art.

It was a further object of the invention to provide a foamed article which has high stability and high tear resistance coupled with high skin compatibility and a minimum amount of toxic or cytotoxic constituents.

It was a further object to provide a process that enables the production of a foamed article of maximum stability and tear resistance and/or good skin compatibility, especially a non-toxic or non-cytotoxic foamed article.

The present invention firstly provides a foamed article produced by mixing a first polyurethane dispersion (A) with at least one second polyurethane dispersion (B), optionally with addition of further additives, foaming and subsequently drying the mixture, wherein the polyurethane dispersion (A) is obtainable by preparing
A) isocyanate-functional prepolymers from
   A1) organic polyisocyanates
   A2) polymeric polyols having number-average molecular weights of 400 to 8000 g/mol, preferably of 400 to 6000 g/mol, or preferably of 600 to 3000 g/mol, and OH functionalities of 1.5 to 6, preferably of 1.8 to 3, or preferably of 1.9 to 2.1, and
   A3) optionally hydroxyl-functional compounds having molecular weights of 62 to 399 g/mol, and
   A4) optionally isocyanate-reactive, anionic or potentially anionic and optionally nonionic hydrophilizing agents,
      and
B) the free NCO groups thereof are then wholly or partly reacted
   B1) optionally with amino-functional compounds having molecular weights of 32 to 400 g/mol and
   B2) with amino-functional, anionic or potentially anionic hydrophilizing agents
      by chain extension and the prepolymers are dispersed in water before, during or after step B), wherein any potentially anionic groups present are converted to the anionic form by partial or complete reaction with a neutralizing agent, and
   wherein the second polyurethane dispersion (B) is obtainable by reactive conversion of at least the following components:
A. an aliphatic polyisocyanate component having an average isocyanate functionality of ≥1.8 and ≤2.6,
B. a polymeric polyether polyol component;
C. an amino-functional chain extender component having at least 2 isocyanate-reactive amino groups, comprising at least one amino-functional compound C1 that does not have any ionic or ionogenic groups and/or an amino-functional compound C2 that has ionic or ionogenic groups,
D. optionally further hydrophilizing components other than C2.,
E. optionally hydroxyl-functional compounds having a molecular weight of 62 to 399 mol/g,
F. optionally further polymeric polyols other than B.,
G. a compound having exactly one isocyanate-reactive group or a compound having more than one isocyanate-reactive group where only one of the isocyanate-reactive groups reacts with the isocyanate groups present in the reaction mixture under the reaction conditions chosen, and
H. optionally an aliphatic polyisocyanate component having an average isocyanate functionality of ≥2.6 and ≤4,
where components B. and F. together contain ≤30% by weight of component F., preferably ≤20% by weight of component F., or preferably ≤10% by weight of component F., based on the total mass of components B. and F.

The polyurethane dispersions (A) and (B), in terms of their composition, differ at least in the selection or amount of at least one component. Polyurethane dispersions (A) and (B) are consequently chemically different at least in a portion of the respective polymer and are distinguishable from one another.

The constituents of the dispersions (A) and (B) and all other components for formation of the foamed article are preferably of low to zero cytotoxicity. What is meant by low to zero cytotoxicity in accordance with the invention is that the foamed article and preferably all constituents of the formed article have a viability of ≥70%, preferably ≥80%, or a classification of 0 to 2 (“no to mild”, which means low to zero cytotoxicity) in a cytotoxicity test according to DIN ISO 10993-5:2009-10.

### Polyurethane Dispersion (A)

In order to achieve anionic hydrophilization in the preparation of the polyurethane dispersion (A), preference is given to using, in A4) and/or B2), hydrophilizing agents that have at least one group reactive toward NCO groups, such as amino, hydroxyl or thiol groups in the case of A4) and amino groups in the case of B2), and additionally have —COO— or —SO₃— or —PO₃²⁻ as anionic groups or the wholly or partly protonated acid forms thereof as potentially anionic groups.

Preferred aqueous anionic polyurethane dispersions (A) have a low level of hydrophilic anionic groups, preferably of 2 to 200 milliequivalents, more preferably of less than 10 to 100 milliequivalents, per 100 g of solid resin.

In order to achieve good sedimentation stability, the number average particle size of the polyurethane dispersions (A) is preferably less than 750 nm, more preferably less than 500 nm, determined by means of laser correlation spectroscopy.

The ratio of NCO groups in the compounds from component A1) to NCO-reactive groups, such as amino, hydroxyl or thiol groups, in the compounds of components A2) to A4) in the preparation of the NCO-functional prepolymer is 1.05 to 3.5, preferably 1.2 to 3.0 and more preferably 1.3 to 2.5.

The amino-functional compounds in stage B) are used in such an amount that the equivalents ratio of isocyanate-reactive amino groups of these compounds to the free isocyanate groups of the prepolymer is 40% to 150%, preferably 50% to 125%, more preferably 60% to 100%.

Suitable polyisocyanates of component A1) are the aromatic, araliphatic, aliphatic or cycloaliphatic polyisocyanates having an NCO functionality of ≥2 that are known per se to those skilled in the art.

Examples of such suitable polyisocyanates include butylene 1,4-diisocyanate, hexamethylene 1,6-diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or their mixtures of any isomer content, cyclohexylene 1,4-diisocyanate, phenylene 1,4-diisocyanate, tolylene 2,4- and/or 2,6-diisocyanate, naphthylene 1,5-diisocyanate, diphenylmethane 2,2′- and/or 2,4′- and/or 4,4′-diisocyanate, 1,3- and/or 1,4-bis(2-isocyanatoprop-2-yl)benzene (TMXDI), 1,3-bis (isocyanatomethyl)benzene (XDI), and alkyl 2,6-diisocyanatohexanoates (lysine diisocyanates) having C1-C8-alkyl groups.

As well as the aforementioned polyisocyanates, it is also possible to use proportions of modified diisocyanates having uretdione, isocyanurate, urethane, allophanate, biuret, iminooxadiazinedione and/or oxadiazinetrione structure, and unmodified polyisocyanate having more than 2 NCO groups per molecule, for example 4-isocyanatomethyloctane 1,8-diisocyanate (nonane triisocyanate) or triphenylmethane 4,4′,4″-triisocyanate.

These are preferably polyisocyanates or polyisocyanate mixtures of the aforementioned type that have exclusively aliphatically and/or cycloaliphatically bonded isocyanate groups and an average NCO functionality in the mixture of 2 to 4, preferably 2 to 2.6, and more preferably 2 to 2.4.

Particular preference is given to using, in A1), hexamethylene 1,6-diisocyanate, isophorone diisocyanate, the isomeric bis(4,4′-isocyanatocyclohexyl)methanes, and mixtures thereof.

What are used in A2) are polymeric polyols having a number-average molecular weight Mn of 400 to 8000 g/mol, preferably of 400 to 6000 g/mol and more preferably of 600 to 3000 g/mol. These preferably have an OH functionality of 1.5 to 6, more preferably of 1.8 to 3, most preferably of 1.9 to 2.1.

Such polymeric polyols are the following, which are known per se in polyurethane coating technology: polyester polyols, polyacrylate polyols, polyurethane polyols, polycarbonate polyols, polyether polyols, polyester polyacrylate polyols, polyurethane polyacrylate polyols, polyurethane polyester polyols, polyurethane polyether polyols, polyurethane polycarbonate polyols and polyester polycarbonate polyols. These may be used in A2) individually or in any desired mixtures with one another.

Such polyester polyols are, for example, the conventional polycondensates of di- and optionally tri- and tetraols and di- and optionally tri- and tetracarboxylic acids or hydroxycarboxylic acids or lactones. Rather than the free polycarboxylic acids, it is also possible to use the corresponding polycarboxylic anhydrides or corresponding polycarboxylic esters of lower alcohols for preparation of the polyesters.

Examples of suitable diols are ethylene glycol, butylene glycol, diethylene glycol, triethylene glycol, polyalkylene glycols such as polyethylene glycol, and also propane-1,2-diol, propane-1,3-diol, butane-1,3-diol, butane-1,4-diol, hexane-1,6-diol and isomers, neopentyl glycol or neopentyl glycol hydroxypivalate, preference being given to hexane-1,6-diol and isomers, neopentyl glycol and neopentyl glycol hydroxypivalate. In addition, it is also possible to use polyols such as trimethylolpropane, glycerol, erythritol, pentaerythritol, trimethylolbenzene or tris(hydroxyethyl) isocyanurate.

The dicarboxylic acids used may be phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, 2-methylsuccinic acid, 3,3-diethylglutaric acid and/or 2,2-dimethylsuccinic acid. It is also possible to use the corresponding anhydrides as the acid source.

Provided that the average functionality of the polyol to be esterified is >2, it is additionally also possible to use monocarboxylic acids, such as benzoic acid and hexanecarboxylic acid, as well.

Preferred acids are aliphatic or aromatic acids of the aforementioned type. Particular preference is given to adipic acid, isophthalic acid and optionally trimellitic acid.

Examples of hydroxycarboxylic acids that may be used as co-reactants in the preparation of a polyester polyol having terminal hydroxyl groups include hydroxycaproic acid, hydroxybutyric acid, hydroxydecanoic acid, hydroxystearic acid and the like. Suitable lactones are caprolactone, butyrolactone and homologs. Preference is given to caprolactone.

It is likewise possible in A2) to use polycarbonates having hydroxyl groups, preferably polycarbonate diols, having number-average molecular weights Mn of 400 to 8000 g/mol, preferably 600 to 3000 g/mol. These are obtainable by reacting carbonic acid derivatives, such as diphenyl carbonate, dimethyl carbonate or phosgene, with polyols, preferably diols.

Examples of such diols are ethylene glycol, propane-1,2- and 1,3-diol, butane-1,3- and 1,4-diol, hexane-1,6-diol, octane-1,8-diol, neopentyl glycol, 1,4-bishydroxymethylcyclohexane, 2-methylpropane-1,3-diol, 2,2,4-trimethylpentane-1,3-diol, dipropylene glycol, polypropylene glycols, dibutylene glycol, polybutylene glycols, bisphenol A, and lactone-modified diols of the aforementioned type.

It is preferable when the polycarbonate diol contains 40% to 100% by weight of hexanediol, preference being given to hexane-1,6-diol and/or hexanediol derivatives. Such hexanediol derivatives are based on hexanediol and have not only terminal OH groups but also ester groups or ether groups. Such derivatives are obtainable by reaction of hexanediol with excess caprolactone or by etherification of hexanediol with itself to afford di- or trihexylene glycol.

Instead of or in addition to pure polycarbonate diols, polyether-polycarbonate diols may also be used in A2). The polycarbonates having hydroxyl groups preferably have a linear structure.

It is likewise possible in A2) to use polyether polyols. Suitable examples are the polytetramethylene glycol polyethers known per se in polyurethane chemistry, as obtainable by cationic ring-opening polymerization of tetrahydrofuran.

Likewise suitable polyether polyols are the addition products of styrene oxide, ethylene oxide, propylene oxide, butylene oxides and/or epichlorohydrin on di- or polyfunctional starter molecules that are known per se. Polyether polyols based on the at least proportional addition of ethylene oxide onto di- or polyfunctional starter molecules can also be used as component A4) (nonionic hydrophilizing agents).

Suitable starter molecules that may be used are all compounds known from the prior art, for example water, butyldiglycol, glycerol, diethylene glycol, trimethylolpropane, propylene glycol, sorbitol, ethylenediamine, triethanolamine, butane-1,4-diol. Preferred starter molecules are water, ethylene glycol, propylene glycol, butane-1,4-diol, diethylene glycol, and butyldiglycol.

Particularly preferred embodiments of the polyurethane dispersions (A) comprise, as component A2), a mixture of polycarbonate polyols and polytetramethylene glycol polyols, where the proportion of polycarbonate polyols in the mixture is 0% to 80% by weight and the proportion of polytetramethylene glycol polyols is 100% to 20% by weight. Preference is given to a proportion of 50% to 100% by weight of polytetramethylene glycol polyols and a proportion of 0% to 50% by weight of polycarbonate polyols. Particular preference is given to a proportion of 75% to 100% by weight of polytetramethylene glycol polyols and a proportion of 0% to 25% by weight of polycarbonate polyols, in each case with the proviso that the sum total of the percentages by weight of the polycarbonate polyols and polytetramethylene glycol polyols is 100% and the proportion of the sum total of the polycarbonate polyols and polytetramethylene glycol polyether polyols in component A2) is at least 50% by weight, preferably 60% by weight and more preferably at least 70% by weight.

The compounds of component A3) have molecular weights of 62 to 400 g/mol.

In A3), it is possible to use polyols of the stated molecular weight range that have up to 20 carbon atoms, such as ethylene glycol, diethylene glycol, triethylene glycol, propane-1,2-diol, propane-1,3-diol, butane-1,4-diol, 1,3-butylene glycol, cyclohexanediol, 1,4-cyclohexanedimethanol, 1,6-hexanediol, neopentyl glycol, hydroquinone dihydroxyethyl ether, bisphenol A (2,2-bis(4-hydroxyphenyl)propane), hydrogenated bisphenol A, (2,2-bis(4-hydroxycyclohexyl)propane), trimethylolpropane, glycerol, pentaerythritol, and any desired mixtures thereof with one another.

Also suitable are ester diols of the stated molecular weight range, such as α-hydroxybutyl ε-hydroxycaproate, w-hydroxyhexyl γ-hydroxybutyrate, β-hydroxyethyl adipate or bis(β-hydroxyethyl) terephthalate.

In addition, it is also possible in A3) to use monofunctional, isocyanate-reactive, hydroxyl-containing compounds. Examples of such monofunctional compounds are ethanol, n-butanol, ethylene glycol monobutyl ether, diethylene glycol monomethyl ether, ethylene glycol monobutyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol. Preferred compounds of component A3) are hexane-1,6-diol, butane-1,4-diol, neopentyl glycol and trimethylolpropane.

Anionically or potentially anionically hydrophilizing compounds in component A4) are all compounds that have at least one isocyanate-reactive group, such as a hydroxyl group, and at least one functionality such as —COO⁻M⁺, —SO₃⁻M⁺, —PO(O⁻M⁺)₂ where M⁺ is, for example, a metal cation, H⁺, NH₄⁺, NHR₃⁺, where R can be in each case a C1-C12 alkyl radical, C5-C6 cycloalkyl radical and/or a C2-C4 hydroxyalkyl radical that enters into a pH-dependent dissociation equilibrium on interaction with aqueous media and may be negatively charged or uncharged in this manner. Suitable anionically or potentially anionically hydrophilizing compounds are mono- and dihydroxycarboxylic acids, mono- and dihydroxysulfonic acids, and mono- and dihydroxyphosphonic acids, and salts thereof. Examples of such anionic or potentially anionic hydrophilizing agents are dimethylolpropionic acid, dimethylolbutyric acid, hydroxypivalic acid, malic acid, citric acid, glycolic acid, lactic acid and the propoxylated adduct of 2-butenediol and NaHSO3, as described in DE-A 2 446 440, pages 5-9, formula I-III. Preferred anionic or potentially anionic hydrophilizing agents in component A4) are those of the aforementioned type that have carboxylate/carboxylic acid groups and/or sulfonate groups.

Particularly preferred anionic or potentially anionic hydrophilizing agents are those that contain carboxylate or carboxylic acid groups as ionic or potentially ionic groups, such as dimethylolpropionic acid, dimethylolbutyric acid and hydroxypivalic acid, or salts thereof.

Suitable nonionically hydrophilizing compounds in component A4) are, for example, polyoxyalkylene ethers having at least one hydroxy or amino group, preferably at least one hydroxy group.

Examples are the monohydroxyl-functional polyalkylene oxide polyether alcohols having a statistical average of 5 to 70, preferably 7 to 55, ethylene oxide units per molecule, as obtainable in a manner known per se by alkoxylation of suitable starter molecules (described for example in Ullmanns Encyclopädie der technischen Chemie [Ullmann's Encyclopedia of Industrial Chemistry], 4th edition, volume 19, Verlag Chemie, Weinheim p. 31-38).

These are either pure polyethylene oxide ethers or mixed polyalkylene oxide ethers and they contain at least 30 mol %, preferably at least 40 mol %, based on all alkylene oxide units present, of ethylene oxide units.

Preferred polyethylene oxide ethers of the aforementioned type are monofunctional mixed polyalkylene oxide polyethers having 40 to 100 mol % of ethylene oxide units and 0 to 60 mol % of propylene oxide units.

Preferred nonionically hydrophilizing compounds of component A4) are those of the aforementioned type, where these are block (co)polymers that are prepared by blockwise addition of alkylene oxides onto suitable starters.

Suitable starter molecules for such nonionic hydrophilizing agents are saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or olein alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and heterocyclic secondary amines such as morpholine, pyrrolidine, piperidine or 1H-pyrazole. Preferred starter molecules are saturated monoalcohols of the abovementioned type. It is particularly preferable to use diethylene glycol monobutyl ethers or n-butanol as starter molecules.

Alkylene oxides suitable for the alkoxylation reaction are especially ethylene oxide and propylene oxide, which can be used in the alkoxylation reaction in any sequence or else in a mixture.

Components B1) that may be used include di- or polyamines such as ethylene-1,2-diamine, 1,2- and 1,3-diaminopropane, 1,4-diaminobutane, 1,6-diaminohexane, isophoronediamine, isomer mixture of 2,2,4- and 2,4,4-trimethylhexamethylenediamine, 2-methylpentamethylenediamine, diethylenetriamine, triaminononane, xylylene-1,3-diamine and -1,4-diamine, α,α,α′,α′-tetramethylxylylene-1,3-diamine and -1,4-diamine, and 4,4-diaminodicyclohexylmethane and/or dimethylethylenediamine. It is likewise possible, albeit less preferred, to use hydrazine and hydrazides such as adipic dihydrazide.

In addition, components B1) used may also be compounds that have not only a primary amino group but also secondary amino groups, or not only an amino group (primary or secondary) but also OH groups. Examples thereof are primary/secondary amines such as diethanolamine, 3-amino-1-methylaminopropane, 3-amino-1-ethylaminopropane, 3-amino-1-cyclohexylaminopropane, 3-amino-1-methylaminobutane, alkanolamines such as N-aminoethylethanolamine, ethanolamine, 3-aminopropanol, neopentanolamine.

Furthermore, components B1) used may also be monofunctional isocyanate-reactive amine compounds, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide amines formed from diprimary amines and monocarboxylic acids, monoketimines of diprimary amines, primary/tertiary amines, such as N,N-dimethylaminopropylamine.

Preferred compounds of component B1) are ethylene-1,2-diamine, 1,4-diaminobutane and isophoronediamine Particular preference is given to using mixtures of the aforementioned diamines of component B1), especially mixtures of ethylene-1,2-diamine and isophoronediamine, and mixtures of 1,4-diaminobutane and isophoronediamine.

Anionically or potentially anionically hydrophilizing compounds in component B2) are all compounds that have at least one amino group and at least one functionality such as —COO⁻M⁺, —SO₃⁻M⁺, —PO(O⁻M⁺)₂. where M⁺ is, for example, a metal cation, H⁺, NH₄⁺, NHR₃⁺, where R can be in each case a C1-C12 alkyl radical, C5-C6 cycloalkyl radical and/or a C2-C4 hydroxyalkyl radical that enters into a pH-dependent dissociation equilibrium on interaction with aqueous media and may be negatively charged or uncharged in this manner.

Suitable anionically or potentially anionically hydrophilizing compounds are mono- and diaminocarboxylic acids, mono- and diaminosulfonic acids, and mono- and diaminophosphonic acids, and salts thereof. Examples of such anionic or potentially anionic hydrophilizing agents are N-(2-aminoethyl)-β-alanine, 2-(2-aminoethylamino)ethanesulfonic acid, ethylenediaminepropyl- or -butylsulfonic acid, propylene-1,2- or -1,3-diamine-β-ethylsulfonic acid, glycine, alanine, taurine, lysine, 3,5-diaminobenzoic acid and the addition product of IPDA and acrylic acid (EP-A 0 916 647, example 1). It is also possible to use cyclohexylaminopropanesulfonic acid (CAPS) from WO-A 01/88006 as anionic or potentially anionic hydrophilizing agent.

Hydrophilization can also be accomplished using mixtures of anionic or potentially anionic hydrophilizing agents and nonionic hydrophilizing agents.

In a preferred embodiment for production of the specific polyurethane dispersions (A), components A1) to A4) and B1) to B2) are used in the following amounts, where the individual amounts always add up to 100% by weight:

5% to 40% by weight of component A1),
55% to 90% by weight of A2),
0% to 20% by weight of the sum total of components A3) and B1),
0.1% to 25% by weight of the sum total of components A4) and B2), with use of 0.1% to 10% by weight of anionic or potentially anionic hydrophilizing agents from A4) and/or B2), based on the total amounts of components A1) to A4) and B1) to B2).

In a particularly preferred embodiment for preparation of the specific polyurethane dispersions (A), components A1) to A4) and B1) to B2) are used in the following amounts, where the individual amounts always add up to 100% by weight:

5% to 35% by weight of component A1),
60% to 90% by weight of A2),
0.5% to 15% by weight of the sum total of components A3) and B1),
0.5% to 15% by weight of the sum total of components A4) and B2), with use of 0.5% to 7% by weight of anionic or potentially anionic hydrophilizing agents from A4) and/or B2), based on the total amounts of components A1) to A4) and B1) to B2).

The production of the anionically hydrophilized polyurethane dispersions (A) can be performed in one or more stages in homogeneous phase or, in the case of a multistage reaction, partially in disperse phase. Complete or partial performance of polyaddition from A1) to A4) is followed by a dispersing, emulsifying or dissolving step. This is optionally followed by a further polyaddition or modification in disperse phase.

Any prior art method can be used, for example the prepolymer mixing method, the acetone method or the melt dispersing method. Preference is given to using the acetone method.

For production by the acetone method, it is customary to form an initial charge including all or some of constituents A2) to A4) and the polyisocyanate component A1) for preparation of an isocyanate-functional polyurethane prepolymer, and optionally to dilute them with a solvent that is water-miscible but inert toward isocyanate groups, and heat them to temperatures in the range from 50° C. to 120° C. The isocyanate addition reaction can be accelerated using the catalysts known in polyurethane chemistry.

Suitable solvents are the customary aliphatic, keto-functional solvents, such as acetone, 2-butanone, which can be added not just at the start of the preparation but optionally also in portions at a later stage. Preference is given to acetone and 2-butanone.

Other solvents such as xylene, toluene, cyclohexane, butyl acetate, methoxypropyl acetate, N-methylpyrrolidone, N-ethylpyrrolidone, or solvents having ester or ether units, may be used additionally and may be wholly or partly distilled off, or remain entirely in the dispersion in the case of N-methylpyrrolidone and N-ethylpyrrolidone. Preference however is given to using no other solvents apart from the aliphatic, keto-functional solvents mentioned.

Subsequently, any constituents of A1) to A4) not yet added at the start of the reaction are added.

In the preparation of the polyurethane prepolymer from A1) to A4), the molar ratio of isocyanate groups to isocyanate-reactive groups is 1.05 to 3.5, preferably 1.2 to 3.0 and more preferably 1.3 to 2.5.

Components A1) to A4) are converted partly or fully to the prepolymer, but preferably fully. Polyurethane prepolymers containing free isocyanate groups are thus obtained in neat form or in solution.

In the neutralization step, partial or complete conversion of potentially anionic groups to anionic groups is accomplished using bases such as tertiary amines, e.g. trialkylamines having 1 to 12 and preferably 1 to 6 carbon atoms, more preferably 2 to 3 carbon atoms, in each alkyl radical, or alkali metal bases such as the corresponding hydroxides.

Examples thereof are trimethylamine, triethylamine, methyldiethylamine, tripropylamine, N-methylmorpholine, methyldiisopropylamine, ethyldiisopropylamine and diisopropylethylamine. The alkyl radicals may, for example, also bear hydroxyl groups, as in the case of the dialkylmonoalkanolamines, alkyldialkanolamines and trialkanolamines. Usable neutralizing agents may also include inorganic bases such as aqueous ammonia solution or sodium or potassium hydroxide.

Preference is given to ammonia, triethylamine, triethanolamine, dimethylethanolamine or diisopropylethylamine, and sodium hydroxide and potassium hydroxide, particular preference to sodium hydroxide and potassium hydroxide.

The molar amount of the bases is between 50 and 125 mol %, preferably between 70 and 100 mol %, of the molar amount of the acid groups to be neutralized. The neutralization can also be effected simultaneously with the dispersing when the dispersion water already contains the neutralizing agent.

Thereafter, in a further process step, if this has not yet been done or has been done only partially, the prepolymer obtained is dissolved using aliphatic ketones such as acetone or 2-butanone.

In the chain extension of stage B), NH₂- and/or NH-functional components are partly or fully reacted with the isocyanate groups still remaining in the prepolymer. It is preferable when the chain extension/termination is carried out prior to the dispersing in water.

Chain termination is typically accomplished using amines B1) having an isocyanate-reactive group, for example methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, or suitable substituted derivatives thereof, amide amines formed from diprimary amines and monocarboxylic acids, monoketimines of diprimary amines, primary/tertiary amines, such as N,N-dimethylaminopropylamine.

If partial or complete chain extension is accomplished using anionic or potentially anionic hydrophilizing agents according to definition B2) that have NH₂ groups or NH groups, the chain extension of the prepolymers preferably precedes the dispersing.

The aminic components B1) and B2) may optionally be used in water- or solvent-diluted form in the process of the invention, individually or in mixtures, any sequence of addition being possible in principle.

When water or organic solvents are included as a diluent, the diluent content in the component for chain extension used in B) is preferably in the range from 70% to 95% by weight.

The dispersing preferably follows the chain extension. To this end, the dissolved and chain-extended polyurethane polymer is either introduced into the dispersion water, optionally under high shear, for example vigorous stirring, or, conversely, the dispersion water is stirred into the chain-extended polyurethane polymer solutions. It is preferable when the water is added to the dissolved, chain-extended polyurethane polymer.

The solvent still present in the dispersions after the dispersion step is typically then removed by distillation. Removal even during the dispersing is likewise possible.

The residual content of organic solvents in the polyurethane dispersions (A) is typically less than 1.0% by weight, preferably less than 0.5% by weight, based on the overall dispersion.

The pH of the polyurethane dispersions (A) that are essential to the invention is typically less than 9.0, preferably less than 8.5, more preferably less than 8.0, and is most preferably 6.0 to 7.5.

The solids content of the polyurethane dispersions (A) is preferably 35% to 70% by weight, more preferably 40% to 65% by weight, even more preferably 45% to 60% by weight and especially 45% to 55% by weight.

The amount of anionic or potentially anionic groups on the particle surface, measured via an acid-base titration, is generally 2 to 500 mmol, preferably 30 to 400 mmol, per 100 grams of solids.

### Polyurethane Dispersion (B)

The preparation of the polyurethane dispersion (B) involves first forming a prepolymer containing isocyanate groups, which is chain-extended with amino groups. The use of compounds containing amino groups in the production of the polyurethane dispersion (B) may also give rise to urea groups, which is the reason why the polyurethane present in the polyurethane dispersion (B) is also called polyurethaneurea below, and therefore the polyurethane dispersion (B) is also referred to as polyurethaneurea dispersion (B).

Ionogenic groups in association with the polyurethaneurea dispersion (B) are understood to mean those functional groups that are capable of forming ionic groups, for example by neutralization with a base.

Component A. may be any polyisocyanate that the person skilled in the art would use for the purpose.

Polyisocyanates suitable with preference as component A. are especially the aliphatic polyisocyanates known per se to the person skilled in the art that have an average isocyanate functionality of ≥1.8 and ≤2.6. The term aliphatic also includes cycloaliphatic and/or araliphatic polyisocyanates.

Average isocyanate functionality is understood to mean the average number of isocyanate groups per molecule.

Preferred polyisocyanates are those in the molecular weight range from 140 to 336 g/mol. These are more preferably selected from the group consisting of: 1,4-diisocyanatobutane (BDI), pentane 1,5-diisocyanate (PDI), 1,6-diisocyanatohexane (HDI), 1,3-bis(isocyanatomethyl)benzene (xylylene 1,3-diisocyanate, XDI), 1,4-bis(isocyanatomethyl)benzene (xylylene 1,4-diisocyanate, XDI), 1,3-bis(1-isocyanato-1-methyl-ethyl)benzene (TMXDI), 1,4-bis(1-isocyanato-1-methylethyl)benzene (TMXDI), 4-isocyanatomethyloctane 1,8-diisocyanate (trisisocyanatononane (TIN)), 2-methyl-L5-diisocyanatopentane, 1,5-diisocyanato-2,2-dimethylpentane, 2,2,4- or 2,4,4-trimethyl-1,6-diisocyanatohexane, 1,10-diisocyanatodecane, and the cycloaliphatic diisocyanates 1,3- or 1,4-diisocyanatocyclohexane, 1,4-diisocyanato-3,3,5-trimethylcyclohexane, 1,3-diisocyanato-2(4)-methylcyclohexane, 1-isocyanato-3,3,5-trimethyl-5-isocyanatomethylcyclohexane (isophorone diisocyanate, IPDI), 1-isocyanato-1-methyl-4(3)isocyanatomethylcyclohexane, 1,8-diisocyanato-p-menthane, 4,4′-diisocyanato-1,1′-bi(cyclohexyl), 4,4′-diisocyanato-3,3′-dimethyl-1,1′-bi(cyclohexyl), 4,4′-diisocyanato-2,2′,5,5′-tetramethyl-1,1′-bi(cyclohexyl), 4,4′- and/or 2,4′-diisocyanatodicyclohexylmethane, 4,4′-diisocyanato-3,3′-dimethyldicyclohexylmethane, 4,4′-diisocyanato-3,3′,5,5′-tetramethyldicyclohexylmethane, 1,3-diisocyanatoadamantane, and 1,3-dimethyl-5,7-diisocyanatoadamantane or any mixtures of such isocyanates. The polyisocyanates are most preferably selected from butylene 1,4-diisocyanate, pentamethylene 1,5-diisocyanate (PDI), hexamethylene 1,6-diisocyanate (HDI), isophorone diisocyanate (IPDI), 2,2,4- and/or 2,4,4-trimethylhexamethylene diisocyanate, the isomeric bis(4,4′-isocyanatocyclohexyl)methanes or mixtures thereof with any isomer content (H12-MDI), cyclohexylene 1,4-diisocyanate, 4-isocyanatomethyloctane 1,8-diisocyanate (nonane triisocyanate) and alkyl 2,6-diisocyanatohexanoates (lysine diisocyanates) having C1-C8-alkyl groups.

As well as the aforementioned polyisocyanates, it is also possible to use modified diisocyanates having an average isocyanate functionality of ≥2 and ≤2.6, with uretdione, isocyanurate, urethane, allophanate, biuret, iminooxadiazinedione or oxadiazinetrione structure, and mixtures of proportions of these and/or the above.

Preference is given to polyisocyanates or polyisocyanate mixtures of the aforementioned type having exclusively aliphatically or cycloaliphatically bonded isocyanate groups or mixtures of these and an average NCO functionality of the mixture of ≥1.8 and ≤2.6 and more preferably ≥2.0 and ≤2.4.

More preferably, the organic polyisocyanate component A. contains an aliphatic or cycloaliphatic polyisocyanate selected from HDI, IPDI and/or H12-MDI or the modification products thereof, most preferably selected from HDI and/or IPDI.

In a preferred variant, IPDI and HDI are present in a mixture as component A.

The weight ratio of IPDI:HDI here is preferably in the range from 1.05 to 10, more preferably in the range from 1.1 to 5, and most preferably in the range from 1.1 to 1.5.

Preferably, the polyurethaneurea used in accordance with the invention is prepared using ≥5% and ≤40% by weight of component A. and more preferably ≥10% and ≤35% by weight of component A., based in each case on the total mass of the polyurethaneurea.

Preferably, the polyurethaneurea is also prepared using component H., an aliphatic polyisocyanate component having an average isocyanate functionality (average number of isocyanate groups per molecule) of >2.6 and ≤4, preferably ≥2.8 and ≤3.8. Component H. is preferably used here in a mixture with component A.

Particularly suitable components H. are oligomeric diisocyanates having a functionality of >2.6 and ≤4, preferably ≥2.8 and ≤3.8, with isocyanurate, urethane, allophanate, biuret, iminooxadiazinedione or oxadiazinetrione structure. Most preferably, H. contains isocyanurate structures.

The organic polyisocyanate component H. preferably consists of an aliphatic or cycloaliphatic polyisocyanate oligomer based on HDI, IPDI and/or H12-MDI, most preferably based on HDI.

The molar ratio of the NCO groups from component A. to component H. is preferably 100:0.5 to 100:50, more preferably 100:2 to 100:15 and most preferably 100:3 to 100:8.

Preferably, the polyurethaneurea used in accordance with the invention is prepared using ≥0% and ≤10% by weight of component H. and more preferably ≥0.1% and ≤3% by weight of component H., based in each case on the total mass of the polyurethaneurea.

The polymeric polyether polyols used in accordance with the invention as component B. preferably have number-average molecular weights of ≥500 and ≤8000 g/mol, determined via gel permeation chromatography versus polystyrene standard in tetrahydrofuran at 23° C., more preferably ≥400 and ≤6000 g/mol, and especially preferably ≥600 and ≤3000 g/mol, and/or OH functionalities of preferably ≥1.5 and ≤6, more preferably ≥1.8 and ≤3, especially preferably ≥1.9 and ≤2.1. The expression “polymeric” polyether polyols here means more particularly that the polyols mentioned have at least three, more preferably at least four, repeat units bonded to one another.

Number-average molecular weight is determined in the context of this application by gel permeation chromatography (GPC) in tetrahydrofuran at 23° C., unless stated otherwise. The procedure here is in accordance with DIN 55672-1: “Gel permeation chromatography, Part 1-Tetrahydrofuran as eluent” (SECurity GPC System from PSS Polymer Service, flow rate 1.0 ml/min; columns: 2×PSS SDV linear M, 8×300 mm, 5 μm; RID detector). Polystyrene samples of known molar mass are used for calibration. The number-average molecular weight is calculated with software support. Baseline points and evaluation limits are fixed according to DIN 55672 Part 1.

Suitable polyether polyols are, for example, the addition products, known per se, of styrene oxide, ethylene oxide, propylene oxide, butylene oxide and/or epichlorohydrin onto di- or polyfunctional starter molecules. Polyalkylene glycols in particular, such as polyethylene glycols, polypropylene glycols and/or polybutylene glycols, are employable, especially with the abovementioned preferred molecular weights. Suitable starter molecules that may be used are all compounds known from the prior art, for example water, butyldiglycol, glycerol, diethylene glycol, trimethylolpropane, propylene glycol, sorbitol, ethylenediamine, triethanolamine, butane-1,4-diol.

Component B. preferably comprises or consists of poly(tetramethylene glycol) polyether polyols.

Suitable poly(tetramethylene glycol) polyether polyols are obtainable, for example, by polymerization of tetrahydrofuran by means of cationic ring opening.

Preferably, component B. contains or consists of a mixture of poly(tetramethylene glycol) polyether polyols, where the poly(tetramethylene glycol) polyether polyols differ in their number-average molecular weights.

According to the invention, the polyurethaneurea is prepared using an amino-functional chain extender component C. having at least 2 isocyanate-reactive amino groups, containing at least one amino-functional compound C1. that does not have any ionic or ionogenic groups and/or an amino-functional compound C2. that has ionic or ionogenic groups.

The amino-functional compounds of the component C. are preferably selected from primary and/or secondary diamines More particularly, the amino-functional compounds C. comprise at least one diamine.

The amino-functional component C. preferably comprises at least one amino-functional compound C2. having ionic and/or ionogenic groups.

The amino-functional component C. preferably comprises both amino-functional compounds C2. having an ionic and/or ionogenic group and amino-functional compounds C1. having no ionic or ionogenic group.

For example, components C1. used may be organic di- or polyamines, for example ethylene-1,2-diamine, 1,2- and 1,3-diaminopropane, 1,4-diaminobutane, 1,6-diaminohexane, isophoronediamine (IPDA), isomeric mixture of 2,2,4- and 2,4,4-trimethylhexamethylenediamine, 2-methylpentamethylenediamine, diethylenetriamine, 4,4-diaminodicyclohexylmethane and/or dimethylethylenediamine or mixtures of at least two of these.

Preferably, component C1. is selected from the group consisting of ethylene-1,2-diamine, bis(4-aminocyclohexyl)methane, 1,4-diaminobutane, IPDA, ethanolamine, diethanolamine and diethylenetriamine or a mixture of at least two of these.

Component C1. preferably contains ≥75 mol %, more preferably ≥80 mol %, even more preferably ≥80 mol %, further preferably ≥95 mol % and still further preferably 100 mol % of ethylene-1,2-diamine or IPDA or a mixture of ethylene-1,2-diamine and IPDA, where the sum total of the two amines in relation to the total amount of C1. is preferably within the ranges mentioned. Component C1. preferably contains ≥75 mol %, more preferably ≥80 mol %, even more preferably ≥85 mol %, further preferably ≥95 mol % and still further preferably 100 mol % of ethylene-1,2-diamine.

Preferably, the hydrophilizing component C2. comprises at least one anionically hydrophilizing compound. Further preferably, the hydrophilizing component C2. includes an anionically hydrophilizing compound to an extent of at least 80% by weight, or preferably to an extent of at least 90% by weight, based on the total weight of component C2. More preferably, component C2. consists of exclusively anionically hydrophilizing compounds.

Suitable anionically hydrophilizing compounds comprise at least one anionic or ionogenic group that can be converted to an anionic group. Further preferably, suitable anionically hydrophilizing compounds have at least two amino groups and more preferably two amino groups. More preferably, the hydrophilizing component C2. comprises or consists of an anionically hydrophilizing compound having at least one anionic or ionogenic group and at least two amino groups.

Suitable anionically hydrophilizing compounds as component C2., also called hydrophilizing agents C2. hereinafter, preferably comprise a sulfonic acid or sulfonate group, more preferably a sodium sulfonate group. Suitable anionically hydrophilizing compounds as component C2. are especially the alkali metal salts of mono- and diaminosulfonic acids. Examples of such anionic hydrophilizing agents are salts of 2-(2-aminoethylamino)ethanesulfonic acid, N-(propyl or butyl)ethylenediaminesulfonic acid or propylene-1,2- or -1,3-diamine-β-ethylsulfonic acid or mixtures of at least two of these.

Preferred anionic hydrophilizing agents C2, are those that comprise sulfonate groups as ionic groups and two amino groups, such as the salts of 2-(2-aminoethylamino)ethylsulfonic acid and propylene-1,3-diamine-β-ethylsulfonic acid. Very particular preference is given to using 2-(2-aminoethylamino)ethylsulfonic acid or salts thereof as anionic hydrophilizing agent C2.

The anionic group in component C2. may optionally also be a carboxylate or carboxylic acid group. In that case, component C2. is preferably selected from diaminocarboxylic acids. In this alternative embodiment, however, the carboxylic acid-based components C2. have to be used in higher concentrations compared to those components C2. bearing sulfonate or sulfonic acid groups. More preferably, therefore, the polyurethaneurea is prepared using no hydrophilizing compounds bearing exclusively carboxylate groups as anionic groups of component C2.

Preferably, the polyurethaneurea used in accordance with the invention is prepared using within a range of ≥0.1% and ≤10% by weight of component C2. and more preferably within a range of ≥0.5% and ≤4% by weight of component C2., based in each case on the total mass of the polyurethaneurea.

Hydrophilization can also be accomplished using mixtures of anionic hydrophilizing agents C2. and further hydrophilizing agents D. that are different than C2.

Suitable further hydrophilizing agents D. are, for example, non-ionic hydrophilizing compounds D1. and/or hydroxyl-functional ionic or ionogenic, preferably anionic or anionogenic, hydrophilizing agents D2. Preferably, component D. comprises nonionically hydrophilizing components D1.

Suitable hydroxyl-functional ionic or ionogenic hydrophilizing agents as component D2. are, for example, hydroxycarboxylic acids such as mono- and dihydroxycarboxylic acids, such as 2-hydroxyacetic acid, 3-hydroxypropanoic acid, 12-hydroxy-9-octadecanoic acid (ricinoleic acid), hydroxypivalic acid, lactic acid, dimethylolbutyric acid and/or dimethylolpropionic acid or mixtures of at least two of these. Preference is given to hydroxypivalic acid, lactic acid and/or dimethylolpropionic acid, particular preference to dimethylolpropionic acid. Preference is given to using no hydroxyl-functional ionic or ionogenic hydrophilizing agents D2., especially preferably no hydrophilizing agents having carboxylate and hydroxyl groups, for example dimethylolpropionic acid. Preferably, the amount of hydroxyl-functional ionic or ionogenic hydrophilizing agents D2. is present in the polyurethaneurea within a range from 0% to 1% by weight, or preferably within a range from 0% to 0.5% by weight, based on the total mass of the polyurethaneurea.

Suitable nonionically hydrophilizing compounds as component D1. are, for example, polyoxyalkylene ethers having isocyanate-reactive groups, such as hydroxyl, amino or thiol groups. Preference is given to monohydroxy-functional polyalkylene oxide polyether alcohols having a statistical average of 5 to 70, preferably 7 to 55, ethylene oxide units per molecule, as obtainable in a manner known per se by alkoxylation of suitable starter molecules (for example in Ullmanns Encyclopädie der technischen Chemie [Ullmann's Encyclopedia of Industrial Chemistry], 4th edition, volume 19, Verlag Chemie, Weinheim p. 31-38). These are either pure polyethylene oxide ethers or mixed polyalkylene oxide ethers and they contain at least 30 mol %, preferably at least 40 mol %, based on all alkylene oxide units present, of ethylene oxide units.

Preferred nonionic compounds are monofunctional mixed polyalkylene oxide polyethers having 40 to 100 mol % of ethylene oxide units and 0 to 60 mol % of propylene oxide units.

Suitable starter molecules for such nonionic hydrophilizing agents are especially saturated monoalcohols such as methanol, ethanol, n-propanol, isopropanol, n-butanol, isobutanol, sec-butanol, the isomeric pentanols, hexanols, octanols and nonanols, n-decanol, n-dodecanol, n-tetradecanol, n-hexadecanol, n-octadecanol, cyclohexanol, the isomeric methylcyclohexanols or hydroxymethylcyclohexane, 3-ethyl-3-hydroxymethyloxetane or tetrahydrofurfuryl alcohol, diethylene glycol monoalkyl ethers, for example diethylene glycol monobutyl ether, unsaturated alcohols such as allyl alcohol, 1,1-dimethylallyl alcohol or olein alcohol, aromatic alcohols such as phenol, the isomeric cresols or methoxyphenols, araliphatic alcohols such as benzyl alcohol, anisyl alcohol or cinnamyl alcohol, secondary monoamines such as dimethylamine, diethylamine, dipropylamine, diisopropylamine, dibutylamine, bis(2-ethylhexyl)amine, N-methyl- and N-ethylcyclohexylamine or dicyclohexylamine, and heterocyclic secondary amines such as morpholine, pyrrolidine, piperidine or 1H-pyrazole. Preferred starter molecules are saturated monoalcohols of the abovementioned type. It is particularly preferable to use diethylene glycol monobutyl ether, methanol or n-butanol as starter molecules.

Alkylene oxides suitable for the alkoxylation reaction are especially ethylene oxide and propylene oxide, which can be used in the alkoxylation reaction in any sequence or else in a mixture.

The polyurethaneurea used for formation of the polyurethaneurea dispersion (B) preferably contains within a range of ≥0% and ≤20% by weight of component D., preferably within a range of ≥0% and ≤10% by weight of component D. and most preferably within a range of ≥0% and ≤5% by weight of component D., based in each case on the total mass of the polyurethaneurea. In a further preferred embodiment, component D. is not used for preparation of the polyurethaneurea.

As component E. it is optionally possible to use polyols, especially non-polymeric polyols, of said molecular weight range from 62 to 399 mol/g having up to 20 carbon atoms, such as ethylene glycol, diethylene glycol, triethylene glycol, propane-1,2-diol, propane-1,3-diol, butane-1,4-diol, 1,3-butylene glycol, cyclohexanediol, cyclohexane-1,4-dimethanol, hexane-1,6-diol, neopentyl glycol, hydroquinone dihydroxyethyl ether, bisphenol A (2,2-bis(4-hydroxyphenyl)propane), hydrogenated bisphenol A (2,2-bis(4-hydroxycyclohexyl)propane), trimethylolpropane, trimethylolethane, glycerol, pentaerythritol and any desired mixtures thereof with one another.

Preferably, the polyurethaneurea used for formation of the polyurethaneurea dispersion (B) contains ≤10% by weight of component E., preferably ≤5% by weight and more preferably 0% by weight of component E., based in each case on the total mass of the polyurethaneurea. Preferably, the polyurethaneurea includes component E. within a range from 0.1% to 10% by weight, preferably within a range from 0.2% to 8% by weight, preferably within a range from 0.1% to 5% by weight, based in each case on the total mass of the polyurethaneurea. In a further preferred embodiment, component E. is not used for preparation of the polyurethaneurea.

Preferably, the polyurethaneurea used in accordance with the invention is prepared using within a range of ≥0.5% and ≤20% by weight of the sum total of components C1. and any E. or preferably within a range of ≥1% and ≤15% by weight of the sum total of components C1. and any E., based in each case on the total mass of the polyurethaneurea.

As component F. it is possible to use further polymeric polyols other than B.

Preference is given to polymeric polyols not covered by the definition of B. because they are not polyether polyols—for example the following polyols that are known per se in polyurethane coating technology: polyester polyols, polyacrylate polyols, polyurethane polyols, polycarbonate polyols, polyester polyacrylate polyols, polyurethane polyacrylate polyols, polyurethane polyester polyols, polyurethane polycarbonate polyols and polyester polycarbonate polyols.

Preferably, component F. does not comprise polymeric polyols having ester groups, especially not polyester polyols.

According to the invention, components B. and F. together contain ≤30% by weight, preferably ≤10% by weight and more preferably ≤5% by weight of component F., based on the total mass of components B. and F. Most preferably, component F. is not used for preparation of the polyurethaneurea.

Preferably, the polyurethaneurea used in accordance with the invention is prepared using within a range of ≥55% and ≤90% by weight of the sum total of components B. and any F. and more preferably within a range of ≥60% and ≤85% by weight of the sum total of components B. and any F., based in each case on the total mass of the polyurethaneurea.

Component G. comprises compounds having exactly one isocyanate-reactive group or compounds having more than one isocyanate-reactive group, where only one of the isocyanate-reactive groups reacts with the isocyanate groups present in the reaction mixture under the reaction conditions chosen.

Component G. differs at least in one property from the other isocyanate-reactive groups, especially of component B., that are used for production of the polyurethane dispersion (B). Preferably, component G. differs from the other isocyanate-reactive groups, especially of component B., on account of its chemical composition.

The isocyanate-reactive groups of component G. may be any functional group that can react with an isocyanate group, for example hydroxyl groups, thiol groups or primary and secondary amino groups.

Isocyanate-reactive groups in the context of the invention are especially preferably primary or secondary amino groups that react with isocyanate groups to form urea groups. As well as the amino group, the compounds of component G. may also have other groups that are isocyanate-reactive in principle, such as OH groups, where just one of the isocyanate-reactive groups reacts with the isocyanate groups present in the reaction mixture under the reaction conditions chosen. This can be effected, for example, by reaction of appropriate amino alcohols at relatively low temperatures, for example at 0 to 60° C., preferably at 20 to 40° C. Preference is given here to working in the absence of catalysts that would catalyze the reaction of isocyanate groups with alcohol groups.

Examples of suitable compounds of component G. are primary/secondary amines, such as methylamine, ethylamine, propylamine, butylamine, octylamine, laurylamine, stearylamine, isononyloxypropylamine, dimethylamine, diethylamine, dipropylamine, dibutylamine, N-methylaminopropylamine, diethyl(methyl)aminopropylamine, morpholine, piperidine, diethanolamine, 3-amino-1-methylaminopropane, 3-amino-1-ethylaminopropane, 3-amino-1-cyclohexylaminopropane, 3-amino-1-methylaminobutane, ethanolamine, 3-aminopropanol or neopentanolamine.

Suitable monofunctional compounds are also ethanol, n-butanol, ethylene glycol monobutyl ether, diethylene glycol monomethyl ether, diethylene glycol monobutyl ether, propylene glycol monomethyl ether, dipropylene glycol monomethyl ether, tripropylene glycol monomethyl ether, dipropylene glycol monopropyl ether, propylene glycol monobutyl ether, dipropylene glycol monobutyl ether, tripropylene glycol monobutyl ether, 2-ethylhexanol, 1-octanol, 1-dodecanol, 1-hexadecanol.

Preferably, the polyurethaneurea used in accordance with the invention is prepared using ≥0.1% and ≤20% by weight of component G. and more preferably ≥0.3% and ≤10% by weight of component G., based in each case on the total mass of the polyurethaneurea.

Preferably, component H. is used and the molar ratio of component G. to component H. is preferably 5:1 to 1:5, more preferably 1.5:1 to 1:4 and most preferably 1:1 to 1:3.

Preferably, the polyurethaneureas used in accordance with the invention are prepared using components A. to H. in the following amounts, where the individual amounts always add up to 100% by weight:

5% to 40% by weight of component A.,
55% to 90% by weight of the sum total of components B. and optionally F.,
0.5% to 20% by weight of the sum total of components C1. and optionally E.,
0.1% to 10% by weight of component C2.,
0% to 20% by weight of component D.,
0.1% to 20% by weight of component G. and
0% to 10% by weight of component H.

The foamed article preferably comprises a polyurethaneurea obtainable by reacting at least
A. an aliphatic polyisocyanate component having an average isocyanate functionality of ≥1.8 and ≤2.6, selected from HDI, IPDI and/or H12-MDI or modification products thereof, preferably a mixture of IPDI and HDI,
B. a polymeric polyether polyol component consisting of poly(tetramethylene glycol)polyether polyols (such as (HO—(CH₂—CH₂—CH₂—CH₂—O)ₓ—H)), preferably a polymeric polyether polyol component which is a mixture of at least two poly(tetramethylene glycol) polyether polyols, and where the poly(tetramethylene glycol) polyether polyols differ in their number-average molecular weights,
C. an amino-functional chain extender component having at least 2 isocyanate-reactive primary and/or secondary amino groups, comprising at least one amino-functional compound C1. that does not have any ionic or ionogenic groups and/or an amino-functional compound C2. that has ionic or ionogenic groups,
D. optionally further hydrophilizing components other than C2., which are preferably nonionic ally hydrophilizing components D1.,
E. optionally hydroxyl-functional compounds having a molecular weight of 62 to 399 mol/g,
F. optionally further polymeric polyols other than B.,
G. a compound having exactly one isocyanate-reactive group or a compound having more than one isocyanate-reactive group where only one of the isocyanate-reactive groups reacts with the isocyanate groups present in the reaction mixture under the reaction conditions chosen, where the isocyanate-reactive group is preferably a primary and/or secondary amino and/or hydroxyl group, and
H. optionally an aliphatic polyisocyanate component having an average isocyanate functionality of >2.6 and ≤4, where component H. consists of an aliphatic or cycloaliphatic polyisocyanate oligomer having isocyanurate, urethane, allophanate, biuret, iminooxadiazinedione or oxadiazinetrione structure, preferably based on HDI, IPDI and/or H12-MDI,
   where components B. and F. together contain ≤30% by weight, preferably ≤20% by weight, or preferably ≤10% by weight, of component F., based on the total mass of components B. and F.

The number-average molecular weight of the polyurethaneureas used with preference in accordance with the invention is preferably from ≥2000 to ≤300 000 g/mol, preferably from ≥5000 to ≤150 000 g/mol.

The polyurethaneurea used for production of the foamed article as a basis for the polyurethaneurea dispersion (B) is preferably present in a physiologically acceptable medium. The medium is more preferably water, and the polyurethaneurea is most preferably in the form of an aqueous dispersion (B). In general, alongside other liquid media that are optionally present, for example solvents, water forms the main constituent (≥50% by weight) of the dispersion medium, based on the total amount of the liquid dispersion medium, and possibly even the sole liquid dispersion medium.

The foamed article itself contains the polyurethaneurea per se, which contains only residual amounts of this medium, if any.

Preferably, the polyurethaneurea used is therefore dispersible in water, which means in the context of this invention that the polyurethaneurea at 23° C. can form a sedimentation-stable dispersion in water, especially deionized water.

The polyurethaneureas used for formation of the polyurethaneurea dispersion (B) are preferably obtainable by preparing isocyanate-functional polyurethane prepolymers a) from components A., B. and optionally D. and/or C2., and optionally compounds E. and/or H. (step a), and the free NCO groups thereof are then wholly or partly reacted with the amino-functional chain-extender component C., and also component G. and optionally components D. and H. (step b).

But when component H. is not used until step b), it is preferably added and reacted with the prepolymer A. prior to the addition of component C.

In step b), reaction is preferably effected with a diamine or multiple diamines (component C.) by chain extension, also with addition of the monofunctional component G. as chain terminator to control the molecular weight.

Components A. to H. are defined here as specified above. The abovementioned preferred embodiments are also applicable.

Preferably, in step b), the reaction of the prepolymer a) for preparation of the polyurethaneurea, a mixture of components C1., C2. and G. is reacted. The use of component C1. can result in formation of a high molar mass without a rise in the viscosity of the isocyanate-functional prepolymer prepared beforehand to a degree that would be a barrier to processing. The use of the combination of components C1., C2. and G. can establish an optimal balance between hydrophilicity and chain length.

Preferably, the polyurethane prepolymer a) used has terminal isocyanate groups, meaning that the isocyanate groups are at the chain ends of the prepolymer. More preferably, all chain ends of the prepolymer have isocyanate groups.

The hydrophilizing components C2. and/or D. can be used to control the hydrophilicity of the prepolymer. In addition, further components are of course also significant for the hydrophilicity of the prepolymer, especially also the hydrophilicity of component B.

Preferably, the isocyanate-functional polyurethane prepolymers a) are water-insoluble and non-water-dispersible.

In the context of the invention, the term “water-insoluble, non-water-dispersible polyurethane prepolymer” means more particularly that the water solubility of the prepolymer used in accordance with the invention at 23° C. is less than 10 g/liter, preferably less than 5 g/liter, and the prepolymer at 23° does not result in any sedimentation-stable dispersion in water, especially deionized water. In other words, the prepolymer settles out when an attempt is made to disperse it in water. The water insolubility or lack of dispersibility in water relates to deionized water without addition of surfactants.

Moreover, the polyurethane prepolymer a) used preferably has essentially neither ionic groups nor ionogenic groups (groups capable of forming ionic groups). In the context of the present invention, this means that the proportion of the ionic and/or ionogenic groups, such as anionic groups in particular, such as carboxylate or sulfate, or of cationic groups is less than 15 milliequivalents per 100 g of polyurethane prepolymer a), preferably less than 5 milliequivalents, more preferably less than 1 milliequivalent and most preferably less than 0.1 milliequivalent per 100 g of polyurethane prepolymer a).

In the case of acidic ionic and/or ionogenic groups, the acid number of the prepolymer is appropriately below 30 mg KOH/g of prepolymer, preferably below 10 mg KOH/g of prepolymer. The acid number indicates the mass of potassium hydroxide in milligrams required to neutralize 1 g of the sample to be examined (measurement to DIN EN ISO 211). The neutralized acids, i.e. the corresponding salts, naturally have a zero or reduced acid number. What is crucial here in accordance with the invention is the acid number of the corresponding free acid.

The water-insoluble, non-water-dispersible isocyanate-functional polyurethane prepolymers A. here are preferably obtainable exclusively from components A., B. and optionally D., E. and/or H.

The components are defined here as specified above. The abovementioned preferred embodiments are also applicable.

In an alternative, less preferred embodiment of the invention, the prepolymers a) used for preparation of the polyurethaneurea of the invention are water-soluble or water-dispersible. In this embodiment, the hydrophilizing components D. and/or C2. are used in the preparation of the prepolymer a) in an amount sufficient for the prepolymer to be water-soluble or water-dispersible. The prepolymer a) here preferably has ionic or ionogenic groups.

Suitable hydrophilizing components D. and C2. for this embodiment of the invention are the compounds mentioned above for D. and C2. The hydrophilizing components used are preferably at least the compounds mentioned above under D1. and/or C2.

The polyurethaneureas used for production of the foamed article are preferably dispersed in water before, during or after step b), more preferably during or after step b). In this way, a dispersion of the polyurethaneureas, the polyurethaneurea dispersion (B), is obtained.

The production of the polyurethaneurea dispersions (B) can be conducted here in one or more stage(s) in a homogeneous reaction or in a multistage reaction, partly in disperse phase. Preparation of the prepolymer a) is preferably followed by a dispersion, emulsification or dissolution step. This is optionally followed by a further polyaddition or modification in disperse phase. In this case, the solvent or dispersant suitable for the corresponding prepolymer in each case, for example water or acetone or mixtures thereof, is chosen.

It is possible here to use any methods known from the prior art, for example

prepolymer mixing methods, acetone methods or melt dispersion methods. Preference is given to employing the acetone method.

For preparation by the acetone method, it is customary to form an initial charge including all or some of constituents B., optionally D. and E., and the polyisocyanate component A., optionally in combination with component H., for preparation of an isocyanate-functional polyurethane prepolymer, and optionally to dilute them with a solvent which is water-miscible but inert toward isocyanate groups, and to heat them to temperatures in the range from 50 to 120° C. The isocyanate addition reaction can be accelerated using the catalysts known in polyurethane chemistry.

Suitable solvents are the customary aliphatic, keto-functional solvents, such as acetone, 2-butanone, which can be added not just at the start of the preparation but optionally also in portions at a later stage. Acetone and 2-butanone are preferred and acetone is particularly preferred. The addition of other solvents without isocyanate-reactive groups is also possible, but not preferred.

Subsequently, any constituents of A., B. and optionally H., D. and E. which have not yet been added at the start of the reaction can be metered in.

In the preparation of the polyurethane prepolymers from A., B. and optionally H., D. and E., the molar ratio of isocyanate groups to isocyanate-reactive groups is preferably 1.05 to 3.5, more preferably 1.1 to 3.0 and most preferably 1.1 to 2.5.

Components A., B. and optionally H., D. and E. can be converted fully or partly, but preferably fully, to the prepolymer. In this way, polyurethane prepolymers containing free isocyanate groups can be obtained in neat form or in solution.

If ionogenic groups, for example carboxyl groups, should be present in the prepolymer, these can be converted to ionic groups by neutralization in a further step.

In the neutralization step, for partial or complete conversion of potentially anionic groups to anionic groups, it is possible to use bases such as tertiary amines, e.g. trialkylamines having 1 to 12 and preferably 1 to 6 carbon atoms, more preferably 2 to 3 carbon atoms, in each alkyl radical, or most preferably alkali metal bases such as the corresponding hydroxides.

Usable neutralizing agents are preferably inorganic bases, such as aqueous ammonia solution or sodium hydroxide or potassium hydroxide; particular preference is given to sodium hydroxide and potassium hydroxide.

The molar amount of the bases is preferably between 50 and 125 mol %, more preferably between 70 and 100 mol %, of the molar amount of the acid groups to be neutralized. The neutralization can also be effected simultaneously with the dispersing when the dispersion water already contains the neutralizing agent.

Following the neutralization, in a further process step, if this has not yet been done or has been done only partially, the prepolymer obtained is dissolved using aliphatic ketones such as acetone or 2-butanone.

In the chain extension/termination in stage b), components C., G. and optionally D. are reacted with the isocyanate groups still remaining in the prepolymer. It is preferable when the chain extension/termination is carried out prior to the dispersing in water.

Suitable components C. for chain extension and G. for chain termination have already been listed above. The abovementioned preferred embodiments are also applicable analogously.

If anionic hydrophilizing agents in accordance with definition C2. having NH₂ groups or NH groups are used for chain extension, the chain extension of the prepolymers in step b) is preferably effected prior to the dispersion in water.

The equivalents ratio of NCO-reactive groups in the compounds used for chain extension and chain termination to free NCO groups in the prepolymer is generally between 40% and 150%, preferably between 50% and 110%, more preferably between 60% and 100%.

Components C1., C2. and G. may optionally be used in water- or solvent-diluted form in the process of the invention, individually or in mixtures, any sequence of addition being possible in principle.

When water or organic solvent is included as diluent in step b), the respective diluent content in components C1., C2. and G. used is preferably 40% to 95% by weight.

Dispersion preferably follows after the chain extension and chain termination. For this purpose, the polyurethane polymer that has been dissolved (for example in acetone) and reacted with the amines is either introduced into the dispersion water, optionally under high shear, for example vigorous stirring, or, conversely, the dispersion water is stirred into the chain-extended polyurethane polymer solutions. Preferably, the water is added to the dissolved polyurethane polymer. In this way, the polyurethaneurea dispersion (B) is obtained.

The solvent still present in the dispersions after the dispersion step is typically then removed by distillation. Removal even during the dispersing is likewise possible.

The aqueous polyurethaneurea dispersions (B) obtained preferably have a content of volatile organic compounds (VOCs), for example volatile organic solvents, of less than 10% by weight, more preferably of less than 3% by weight, even more preferably of less than 1% by weight, based on the aqueous polyurethaneurea dispersion. VOCs in the context of this invention are especially organic compounds having an initial boiling point of at most 250° C. at a standard pressure of 101.3 kPa.

In the context of the present invention, the content of volatile organic compounds (VOCs) is especially determined by gas chromatography analysis.

The pH of the aqueous polyurethaneurea dispersion (B) used in accordance with the invention is typically less than 9.0, preferably less than 8.5, and is more preferably between 5.5 and 8.0.

In order to achieve good sedimentation stability, the number-average particle size of the specific polyurethaneurea dispersions is preferably less than 750 nm, more preferably less than 500 nm, determined by means of laser correlation spectroscopy after dilution with deionized water (instrument: Malvern Zetasizer 1000, Malvern Inst. Limited).

The solids content of the polyurethaneurea dispersions (B) is preferably 10% to 70% by weight, more preferably 20% to 60% by weight and most preferably 40% to 60% by weight. The solids contents are ascertained by heating a weighed sample to 125° C. to constant weight. At constant weight, the solids content is calculated by reweighing the sample.

Preferably, these polyurethaneurea dispersions (B) include less than 5% by weight, more preferably less than 0.2% by weight, based on the mass of the dispersions (B), of unbound organic amines.

The polyurethaneurea dispersion (B) used for production of the foamed articles has, at 23° C., at a constant shear rate of 10 s⁻¹, preferably a viscosity of ≥1 and ≤10 000 mPa s, or preferably of ≥10 and ≤5000 mPa s, and more preferably of ≥100 and ≤4000 mPa s. The viscosity is determined as described in the Methods section.

The two polyurethane dispersions (A) and (B) are preferably chemically or physically nonidentical. The two polyurethane dispersions (A) and (B) preferably differ from one another in the level of the molecular masses of the polyurethanes present in (A) and (B). The difference in the average molecular weight Mw (ascertained by GPC with DMAc as eluent) of the polyurethanes of (A) to (B) is preferably within a range from 20:1 to 1.2:1, more preferably from 10:1 to 1.3:1 and most preferably from 5:1 to 1.5:1. The polyurethane polymer of the polyurethane dispersion (A) preferably has an average molecular weight Mw within a range from 150 000 to 1 000 000 g/mol, or preferably from 170 000 to 700 000 g/mol, more preferably from 180 000 to 500 000 g/mol. The polyurethane polymer of the polyurethane dispersion (B) preferably has an average molecular weight Mw within a range from 10 000 to 170 000 g/mol, or preferably from 20 000 to 150 000 g/mol.

As well as the polyurethane dispersions (A) and (B), it is additionally possible to use additives in the form of auxiliaries and additions in the foamed article.

Examples of such auxiliaries and additions are foaming aids such as foam formers and stabilizers, thickeners or thixotropic agents, antioxidants, light stabilizers, emulsifiers, plasticisers, pigments, fillers, dyes, additives for container stabilization, biocides, pH regulators, dispersions and/or leveling aids. According to the desired profile of properties and end use of the foamed articles, there may be ≤30% by weight, preferably ≤20% by weight, or preferably ≤10% by weight, based on total dry matter of the foamed article, of such fillers in the end product. Preference is given to using no dyes or pigments, such that preferably white foams and hence white foamed articles are the result.

Auxiliaries and additions present are preferably foaming aids such as foam formers and stabilizers. Foam stabilizers refer to additions that increase the lifetime of foams by increasing the interfacial viscosity and interfacial elasticity of the foam lamellae, as described in “spektrum.de”, Lexikon der Chemie [Chemical Lexicon]. Usable foam stabilizers are generally any interface-active amphiphilic substances. Particularly suitable examples are commercial compounds such as fatty acid amides, sulfosuccinamides, hydrocarbon sulfonates, hydrocarbon sulfates or fatty acid salts, where the lipophilic radical preferably contains 12 to 24 carbon atoms, amphiphilic organosilicone copolymers, and alkyl polyglycosides obtainable by methods known per se to the person skilled in the art by reaction of relatively long-chain monoalcohols (4 to 22 carbon atoms in the alkyl radical) with mono-, di- or polysaccharides (see, for example, Kirk-Othmer, Encyclopedia of Chemical Technology, John Wiley & Sons, vol. 24, p. 29). Particularly suitable foaming aids are EO/PO block copolymers obtainable by methods known per se to the person skilled in the art by addition of ethylene oxide and propylene oxide onto OH- or NH-functional starter molecules (see, for example, Kirk-Othmer, Encyclopedia of Chemical Technology, John Wiley & Sons, vol. 24, p. 28). In order to improve foam formation, foam stability or the properties of the resulting polyurethane foam, it is possible for further additives to be present in the mixture as well as or as an alternative to the EO/PO block copolymers. Such further additives may in principle be any anionic, nonionic and cationic surfactants known per se, preference being given to anionic and nonionic surfactants, particular preference to nonionic surfactants. Surfactants are substances that lower the surface tension of a liquid or the interfacial tension between two phases. Particular compounds are consequently able to act both as surfactant and as foam stabilizer. However, foam-forming auxiliaries used are especially preferably solely amphiphilic, nonionic EO/PO block copolymers as additives. Preference is given to using 0.5% to 5% by weight, or preferably 1% to 4% by weight, more preferably 1.5% to 3.4% by weight, of foam stabilizer, based on the total mass of polyurethane dispersions (A) and (B). As will be elucidated in detail later on, preference is given to foam stabilizers having minimal to zero cytotoxic activity, which can be ascertained in the form of cell viability in a cytotoxicity test. The foam stabilizers preferably have a cell viability of ≥70% or a classification of 0-2 (no to mild cytotoxicity) in a cytotoxicity test according to DIN ISO 10993-5.

Thickeners used may be commercial thickeners, such as dextrin derivatives, starch derivatives, polysaccharide derivatives such as guar gum, or cellulose derivatives such as cellulose ethers or hydroxyethyl cellulose, fully synthetic organic thickeners based on polyacrylic acids, polyvinylpyrrolidones, poly(meth)acryloyl compounds or polyurethanes (associative thickeners), and inorganic thickeners, such as bentonites or silicas. Particular preference is given to using associative polyurethane thickeners. The amounts of thickener are preferably ≤5% by weight, or preferably within a range from 0.1% to 5% by weight, further preferably from 0.2% to 2% by weight, based on the total mass of the sum total of dispersions (A) and (B). Preference is given to using thickeners having minimal to zero cytotoxic activity, which can be ascertained in the form of cell viability in a cytotoxicity test.

It is further possible with preference to use particular crosslinkers for production of the foamed article, in order, for example, to improve the chemical resistance of the foamed article to bases and acids or solvents. Examples of these are polyisocyanate-based crosslinkers such as Bayhydur® 3100, Baymedix® FP520. The foamed article includes crosslinkers preferably in an amount within a range from 0% to 5% by weight, or preferably from 0.1% to 4.5% by weight, or preferably within a range from 0.5% to 3% by weight, based on the total mass of the foamed article. Likewise possible is addition, incorporation or coating of the foamed article by or with antimicrobial or biological active ingredients that have a positive effect, for example in relation to wound healing and the avoidance of microbial contamination. These active ingredients can be added or incorporated by addition either to the polyurethane dispersion (A) and/or to the polyurethaneurea dispersion (B).

Preferred active ingredients of the aforementioned type are those from the group of the antiseptics, growth factors, protease inhibitors and nonsteroidal antiphlogistics/opiates, or else active ingredients such as, for example, thrombin alpha for local hemostasis.

The active ingredient preferably comprises at least one bacteriostat or a bactericide, most preferably an antiseptic biguanide and/or salt thereof, preferably the hydrochloride.

Examples of mixtures for production of the article of the invention are detailed hereinafter, where the sum total of the figures in percent by weight assumes a value of ≤100% by weight. These mixtures comprise, based on the total mass of the mixture, typically ≥80% to ≤100% by weight of the dispersions (A) and (B) in total, ≥0% to ≤10% by weight of foam auxiliaries, ≥0% to ≤10% by weight of crosslinkers and ≥0% to ≤10% by weight of thickeners.

The mixture preferably comprises, based on the total mass of the mixture, ≥85% to ≤100% by weight of the dispersions (A) and (B) in total, ≥0% to ≤7% by weight of foam auxiliaries, ≥0% to ≤5% by weight of crosslinkers, ≥0% to ≤10% by weight of antiseptics or biocides and ≥0% to ≤5% by weight of thickeners.

The mixture more preferably comprises, based on the total mass of the mixture, ≥89% to ≤100% by weight of the dispersions (A) and (B) in total, ≥0% to ≤6% by weight of foam auxiliaries, ≥0% to ≤4% by weight of crosslinkers and ≥0% to ≤4% by weight of thickeners.

The foamed article is preferably crack-free. What is understood by “crack-free” according to the invention is that at least one, preferably at least two, more preferably all of the following criteria are fulfilled in respect of the foamed article:
i. the foamed article has no cracks having a width of ≥10 mm, preferably of ≥5 mm, more preferably of ≥2 mm, especially preferably of ≥0.5 mm;
ii. the foamed article has no surface cracks deeper than 0.4 mm;
iii. the foamed article has a crack area of ≤6%, preferably ≤3% or preferably ≤0.5%, based on the total area of the foamed article.

Crack width under i., crack depth under ii. and crack area under iii. are each determined by the method as described further down in the test methods.

In a preferred configuration of the foamed article, the foamed article has at least one, preferably at least two, or preferably all of the following properties i. to xiii.:
i. the foamed article has no cracks having a width of ≥2 mm, preferably of ≥1 mm, more preferably of ≥0.5 mm, especially preferably of ≥0.1 mm, the crack width having been determined by the method under test methods;
ii. the foamed article has no surface cracks deeper than 0.4 mm, preferably deeper than 0.2 mm or preferably deeper than 0.1 mm, the crack depth having been determined by the method under test methods;
iii. the foamed article has a crack area of ≤6%, preferably ≤3% or preferably ≤0.5%, based on the total area of the foamed article;
iv. the foamed article has a cell viability of ≥70% or a classification of 0 to 2 (no to mild cytotoxicity) in a cytotoxicity test to DIN ISO 10993-5;
v. the foamed article has a density within a range from 80 to 500 g/l, preferably 100 to 400 g/l, or preferably 120 to 300 g/l;
vi. the foamed article has a thickness (D) within a range from 0.1 to 100 mm, preferably within a range from 0.5 to 50 mm, preferably within a range from 1 to 10 mm;
vii. the foamed article has a tensile stress at break within a range from 100 to 1100 kPa, preferably within a range from 200 to 500 kPa, measured to DIN EN ISO 527-2:2012-06;
viii. the foamed article has an elongation at break within a range from 100% to 500%, preferably within a range from 150% to 400%, measured to DIN EN ISO 527-2:2012-06;
ix. the polyurethaneurea formed from the polyurethane dispersion (B) is amorphous and has a Tg ≤−25° C., preferably ≤−40° C., or preferably ≤−50° C., determined by means of differential scanning calorimetry in accordance with DIN EN 61006, Method A;
x. the foamed article has a melting or softening range of ≤180° C., preferably ≤150° C., at a maximum pressure of 4 bar and has full thermoplastic processability within this range;
xi. the foamed article has an average pore size within a range from 200 to 750 μm, preferably within a range from 300 to 600 μm;
xii. the foamed article has an outer layer, preferably containing a thermoplastic polyurethane or a thermoplastic polyethylene, at least on one surface of the foamed article;
xiii. the foamed article is part of a composite material.

The foamed article preferably has at least the properties i. and ii., more preferably the properties i., and iii. It is further preferable when the foamed article has at least the properties i. and iv., or ii. and iv., or iii. and iv., or most preferably the properties i., ii., iii. and iv.

What is understood by “thermoplastically processible” is that the material can be deformed when heated, or a processing operation such as lamination can take place, without destroying the base structure of the underlying polymer, since, in the case of thermoplastic materials, all that takes place in the case of moderate heating is loss of structure by melting, with subsequent crystallization on cooling.

“Amorphous” in the context of this invention means that the polyurethaneurea, within the temperature range specified in the test method detailed hereinafter, forms only such minor crystalline components, if any, that, by means of the DSC measurements described, it is possible to find only one or more glass transition points T_{g} but no fusion regions having an enthalpy of fusion ≥20 J/g within the temperature range mentioned.

The glass transition temperature T_{g} is determined in the context of this invention by means of dynamic differential calorimetry in accordance with DIN EN 61006, Method A, using a DSC instrument calibrated with indium and lead for determination of T_{g}, by conducting three directly consecutive runs composed of a heating operation from −100° C. to +150° C., at a heating rate of 20 K/min, with subsequent cooling at a cooling rate of 320 K/min, and using the third heating curve to determine the values and determining T_{g} as the temperature at half the height of a glass transition step.

If the polyurethaneurea should be in the form of a dispersion, a special procedure is followed in the sample preparation for the DSC measurements. In the determination of the glass transition temperature T_{g} of dispersions by means of DSC, the T_{g} of the polymer can be masked by the caloric effects of the dispersant (water, neutralizing agent, emulsifier, cosolvent etc.) or distinctly lowered owing to miscibility with the polymer. Therefore, the dispersant, prior to the DSC measurement, is preferably first removed completely by suitable drying, since even small residual amounts of dispersant act as plasticizer and can lower the glass transition temperature as a result. The dispersion is therefore preferably knife-coated onto a glass plate at wet film thickness (WFT) 100 μm, flashed off and then dried in a dry box at RT and 0% relative air humidity (rh) for two days. After this sample preparation, residual moisture in the film can still create a broad endothermic evaporation range in the first heating in the DSC measurement. In order to keep the particular values free of such influences as far as possible, the third heating curve is therefore evaluated.

In a preferred configuration of the foamed article, the weight ratio of the polyurethane dispersion (A) to the polyurethane dispersion (B) is within a range from 1:1 to 5:1, preferably within a range from 1:1 to 4:1, or preferably within a range from 1:1 to 3:1, based on the total mass of the masses of the dispersions (A) and (B).

In a preferred configuration of the foamed article, a film formed from the dispersion (B) by drying has a tensile stress at break of ≤5 MPa, preferably ≤3.5 MPa and more preferably ≤2.5 MPa. The film formed from the dispersion (B) preferably has an elongation at break of ≥1750%, preferably ≥2000%, preferably within a range from ≥1750% to 3000%, more preferably from 2000% to 4000%. The 500% modulus of the film formed from dispersion (B) is preferably ≤2 MPa, or preferably ≤1.5 MPa or preferably ≤1.0 MPa, more preferably within a range from 0.1 to 2 MPa.

For the production of the films required from dispersion (A), dispersion (B) or a mixture of dispersions (A) and (B), a hand coater having a gap width of 200 μm is used to coat the respective dispersion onto a polyolefin-coated release paper, and the coating is dried at 40° C. for 20 min and then at 130° C. for 10 min in an air circulation drying cabinet.

The polyurethaneurea formed from the polyurethane dispersion (B) is preferably amorphous and has a Tg ≤−25° C., preferably ≤−40° C., or preferably ≤−50° C., determined by means of differential scanning calorimetry according to DIN EN 61006, Method A.

It has been found that, surprisingly, it is possible specifically by virtue of the suitable mixture of a foam-forming dispersion (A) with a dispersion (B) from which it is possible to form films that can be described as very soft to produce a foamed article that is free of cracks.

A film formed from dispersion (A) preferably has an elongation at break of 400% to 700%, a tensile stress at break of 20 to 50 MPa, and a 100% modulus of preferably ≤7 MPa, preferably within a range from 2 to 7 MPa. The process for producing the film from dispersion (A) will be described in detail later on.

A film formed from dispersion (A) preferably has an elongation at break within a range from 1% to 50% of the elongation at break of a film formed from dispersion (B). A film formed from dispersion (A) preferably has a tensile stress at break within a range from 5% to 40% of the tensile stress at break of a film formed from dispersion (B). A film formed from dispersion (A) preferably has a 100% modulus within a range from 10% to 50% of the 100% modulus of a film formed from dispersion (B).

The module values and elongations for determination of tensile stress at break and elongation at break were determined in a tensile testing method in accordance with DIN 53504:2009-10 at a testing speed of 200 mm/min and an initial force of 0.05 N/mm².

In a preferred configuration of the foamed article, hydrophilizing agents B2) used are hydrophilizing agents containing sulfone groups. Preferred hydrophilizing agents containing sulfone groups are the sodium or potassium salt of hydroxyethanesulfonic acid.

In a preferred configuration of the foamed article, the dispersion (A) has a solids content of polyurethane of 52% to 65% by weight, preferably 55% to 63% by weight, or preferably 57% to 62% by weight, based on the total mass of the dispersion (A).

In a preferred configuration of the foamed article, component A. or A1) is isophorone diisocyanate (IPDI) and/or hexamethylene diisocyanate (HDI). Further preferably, component A. or A1) is a mixture of IPDI and HDI, preferably in a ratio within a range from 2:1 to 1:2, or preferably within a range from 1.5:1 to 1:1.5.

In a preferred configuration of the foamed article, component B. comprises or consists of poly(tetramethylene glycol) polyether polyols.

In a preferred configuration of the foamed article, component B. comprises or consists of a mixture of at least two poly(tetramethylene glycol) polyether polyols, where the at least two poly(tetramethylene glycol) polyether polyols differ in their number-average molecular weights. Preferably, component B. comprises a mixture of poly(tetramethylene glycol) polyether polyols I having a number-average molecular weight Mₙ within a range from ≥400 and ≤1500 g/mol, more preferably within a range from ≥600 and ≤1200 g/mol, most preferably within a range of 1000 g/mol, and poly(tetramethylene glycol) polyether polyols II having a number-average molecular weight Mₙ within a range from ≥1500 and ≤8000 g/mol, more preferably within a range from ≥1800 and ≤3000 g/mol, most preferably of 2000 g/mol.

The weight ratio of the poly(tetramethylene glycol) polyether polyols I to the poly(tetramethylene glycol) polyether polyols II is preferably in the range from 0.1 to 10, more preferably in the range from 0.2 to 10, most preferably in the range from 1 to 6.

In a preferred configuration of the foamed article, component D. comprises nonionically hydrophilizing components D1. Preferred nonionically hydrophilizing components D1. are nonionic monofunctional mixed polyalkylene oxide polyethers which comprise 40 to 100 mol % of ethylene oxide and 0 to 60 mol % of propylene oxide units, as already described above.

In a preferred configuration of the foamed article, component H. is used, and the molar ratio of component G. to component H. is 5:1 to 1:5, or preferably 4.5:1 to 1:4.5, or preferably 4:1 to 1:4.

In a preferred configuration of the foamed article, the polyurethane dispersion (B) is obtainable by preparing isocyanate-functional polyurethane prepolymers a) from components A., B. and optionally D. and/or C2., and optionally compounds E. and/or H., and the free NCO groups thereof are then wholly or partly reacted with the amino-functional chain-extender component C., and also component G. and optionally components D. and H.

The invention further provides a process for producing a foamed article of the invention, wherein the process comprises at least the following steps:
(V1) mixing the polyurethane dispersion (A) and the polyurethane dispersion (B) to obtain a mixture (M1),
(V2) optionally adding to (M1) at least one interface-active substance, such as a foam stabilizer, that is preferably non-cytotoxic,
(V3) optionally adding to (M1) at least one surfactant (liquid/liquid or liquid/air) that is preferably non-cytotoxic,
(V4) optionally adding to (M1) a thickener, preferably an associative PU thickener, that is preferably non-cytotoxic,
(V5) foaming the mixture (M1) to give a foam, preferably by introducing air into the mixture (M1),
(V6) optionally maturing the foam from step (V2),
(V7) optionally applying the foam formed in step (V5) or (V6) to a substrate,
(V8) drying the foam to form the foamed article.

Dispersions (A) and (B) can be mixed in step (V1) with any equipment and in any conceivable manner that the person skilled in the art would use for homogeneous mixing of polyurethane dispersions to obtain a mixture (M1). Preference is given to performing the mixing in step (V1) in a stainless steel vessel. The mixing in step (V1) preferably takes place over a period of time within a range from 30 seconds to 60 minutes, or preferably within a range from 1 to 30 minutes.

The optional addition of at least one interface-active substance, such as a foam stabilizer in step (V2) and optionally of a surfactant in step (V3) and optionally of a thickener in step (V4), can be effected by any suitable means and in any manner that the person skilled in the art would select for addition of such additives as already described above for the foamed article of the invention to polyurethane dispersions and mixtures thereof.

The at least one interface-active substance is preferably added in step (V2) by adding this substance to one or both of dispersions (A) and (B) before step (V1). The optional addition of the surfactant (V3) and the optional addition of the thickener in step (V4) can likewise be effected into one or both of dispersions (A) and (B) before step (V1).

Alternatively or additionally, the addition of the at least one interface-active substance or the surfactant (V3) and the optional addition of the thickener (V4) can be effected during the mixing in step (V1) or after the mixing in step (V1). It is also possible to introduce multiple interface-active substances and/or multiple surfactants and/or multiple thickeners into the mixture from (V1) in step (V2), (V3) and/or (V4).

The interface-active substances are preferably selected from the group of the additives as described above for the foamed article of the invention. The amounts used of these interface-active substances are also preferably within the ranges as described above for the foamed article of the invention.

The surfactants are preferably selected from the group of the additives as described above for the foamed article of the invention. The amounts used of these surfactants are preferably within the ranges as described above for the foamed article of the invention.

The thickeners are preferably selected from the group of the additives as described above for the foamed article of the invention. The amounts used of these thickeners are preferably within the ranges as described above for the foamed article of the invention.

After performance of steps (V1) and optionally (V2), (V3) and/or (V4), the mixture (M1) is foamed in step (V5) to give a foam. This is preferably effected by introducing air into the mixture (M1). This is typically effected by vigorous stirring or beating of the mixture (like production of whipped cream). In mixing units, preference is given to using a toothed mixing element, and preference is given to applying an air pressure within a range from 3 to 10 bar, preferably from 4 to 8 bar. The foam which is formed in step (V5) is subsequently left to mature for an appropriate period of time in step (V6). An appropriate period of time for maturing is preferably within a range from 1 second to 60 minutes, or preferably within a range from 5 seconds to 30 minutes, or preferably 1 to 10 minutes. The maturing in step (V6) will preferably be effected at room temperature, preferably at 23° C., or will preferably take place at elevated temperature, preferably within a range from 25 to 50° C., or preferably from 30 to 40° C. The maturing in step (V6) in which the foam takes on its final properties is preferably undertaken in the vessel in which the mixing in step (V1) and foaming in step (V5) also takes place. Alternatively, the maturing in step (V6) can also be achieved by means of a longer connecting hose from the mixer to the applicator before the mixture (M1) is applied to a substrate. Such a measure can assure a continuous process in the mixer and in the application. It is also possible to effect a mixture of the two process steps (V6) and (V7).

The substrate to which the foam can optionally be applied in step (V7) may include any material that the person skilled in the art would use as coating substrate for a foam from step (V5). The substrate is preferably selected from the group consisting of a paper, for example a release paper, a belt, for example a conveyor belt made of rubber or a polymer, a metal, a wood surface, a nonwoven, a stone surface, a polymer surface or a glass surface, and a combination of at least two of these. Preference is given to using a release paper, a film or a nonwoven.

After foaming in step (V5) and optionally also after the maturing in step (V6) and/or the applying to a substrate in step (V7), the foam is dried in step (V8). The drying in step (V8) preferably takes place on the substrate from step (V7). For a particularly good drying operation, the foam is applied to the substrate with a metal coater with a layer thickness of 100 μm to 10 mm, preferably 300 μm to 7 mm, more preferably 1 to 5 mm. The drying operation can preferably take place at elevated temperature, preferably within a range from 50 to 130° C., or preferably from 60 to 100° C. Typically, a rising temperature ramp is used in a coating system having various oven zones. The foam is preferably dried for a period of 1 to 60 minutes, or for a period of 10 to 50 minutes, or preferably within a period of 3 to 10 minutes. In order to accelerate the drying operation, it is possible, for example, to pass air heated to the temperature cited above over the foam or to use a microwave oven or an infrared source. After step (V8), the foam preferably has a water content of 0% to 3% by weight, or preferably of 0.2% to 2% by weight. The foam thus dried constitutes the foamed article of the invention.

The invention further provides for use of the foamed article of the invention or of a foamed article produced by the process of the invention as wound dressing, as hygiene article or as wearable patch.

The invention further provides a kit of parts at least including a polyurethane dispersion (A) and a polyurethane dispersion (B), wherein the polyurethane dispersion (A) or the polyurethane dispersion (B) includes an added material (C), wherein the dispersion (A) or (B) including the added material (C) has a cell viability of ≥70%, preferably of ≥75%, or preferably of ≥80%, in a cytotoxicity test according to DIN ISO 10993-5:2009-10.

### Test Methods

### Thickness Measurement:

The determination of layer thickness was ascertained with a compressed air gauge connected to a display from Heidehain (MT25P) to display the layer thickness.

### Density Measurement:

To determine the density, a specimen was punched out with the aid of a punch iron in dimensions of 5×5 cm² (with rounded corners, where the corners have a curve radius of 3 mm). The height was ascertained from the average of a 5-fold determination by means of the test method for thickness measurement described above. For subsequent calculation of the density, the mass of the specimen was determined using a Mettler Toledo X5603 S balance, and together with the volume of the specimen ascertained it was possible to ascertain the density [g/l] of the specimen.

### Crack Area:

For determination of crack areas in a foam, the surface of a piece of foam having dimensions of about 100 cm² was scanned with a flatbed scanner (Epson Perfection V370). In the case of very light-colored foams, scanning was conducted with lid open in order to more clearly distinguish the differences in contrast. In the case of dark-colored foams, the lid was closed in order to form a white background as contrast. For the cracks to be sufficiently illuminated in the scan, the thicknesses of the foam were within a range from 0.1 to 5 mm Subsequently, the scan was binarized by means of the ImageJ software (standard binarization->Image—Adjust—Threshold—Default (0.172)), which means that, above a grayscale value that was chosen at a gray value of higher than 0.172, the area was assessed as being part of a crack and, below 0.172, as being a smooth area. In order to prevent even smaller pores from being detected as a crack, the threshold value for the footprint area for a crack was fixed at ≥0.4 mm². For calculation of the percentage crack area, the crack area detected was expressed relative to the total area.

### Crack Width:

For determination of the crack area of a foam, the surface of the piece of foam having an area of about 100 cm² was scanned with a flatbed scanner (Epson Perfection V370). Subsequently, regions with cracks were defined by the grayscale value as described above, and the crack width was ascertained every 2 mm along the crack length by means of the ImageJ software. Subsequently, the average was formed over the values measured along the crack.

### Crack Depth:

The layer thickness was measured as described above by means of a compressed air gauge and a tip of diameter 3.5 mm. In the case of cracks that had a width of less than 3.5 mm, one side of the crack was cut off before the measurement, such that one flank was accessible for the measurement of crack depth with the compressed air gauge.

### Pore Size:

The average pore size in the foam cross section was determined in accordance with the scientific article “Methods for Cell Structure Analysis of Polyurethane Foams” (Polyurethanes Expo; 2005; pages 453-465; Landers, R., Venzmer J., Boinowitz, T.). The following alterations were made to this in order to determine pore size: scanner type: Epson Perfection V39, resolution: 9600 dpi, 8 bit grayscale levels, Edding 390 black, software: ImageJ Version 1.51n Plugin MorpholibJ.

### EXAMPLE 1: PRODUCTION OF THE POLYURETHANE DISPERSION (A)

1077.2 g of PolyTHF® 2000, 409.7 g of PolyTHF® 1000, 830.9 g of Desmophen® C2200 and 48.3 g of polyether LB 25 were heated to 70° C. in a standard stirring apparatus. Subsequently, at 70° C., a mixture of 258.7 g of hexamethylene diisocyanate and 341.9 g of isophorone diisocyanate was added over 5 min and the mixture was stirred at 120° C. until the NCO value had attained or fallen slightly below the theoretical NCO value. The finished prepolymer was dissolved with 4840 g of acetone and cooled to 50° C., before a solution of 27.4 g of ethylenediamine, 127.1 g of isophoronediamine, 67.3 g of diaminosulfonate and 1200 g of water was added over 10 min. The mixture was stirred for a further 10 min. This was followed by dispersion by addition of 654 g of water. This was followed by removal of the solvent by distillation under vacuum.

The resultant polyurethane dispersion had the following properties:

Solids content: 61.6%
Particle size (LCS): 528 nm

### pH (23° C.): 7.5

### EXAMPLE 2: PRODUCTION OF THE POLYURETHANE DISPERSION (B)

75 g of PolyTHF® 1000 and 350 g of PolyTHF® 2000 were heated to 70° C. Subsequently, a mixture of 33.9 g of hexamethylene diisocyanate, 49.7 g of isophorone diisocyanate and 8.7 g of Desmodur N 3300 (HDI trimer having an NCO content of about 21.8% to DIN EN ISO 11 909) was added, and the mixture was stirred at 100-115° C. until the NCO value had gone below the theoretical value. The finished prepolymer was dissolved with 920 g of acetone at 50° C. and then a solution of 3.2 g of ethylenediamine, 12.9 g of diaminosulfonate, 11.7 g of diethanolamine and 145 g of water was metered in. The mixture was stirred for a further 15 min. This was followed by dispersion by addition of 1080 g of water. Subsequently, the solvent was removed by distillation under reduced pressure, and a storage-stable dispersion was obtained; the solids content was adjusted by addition of water.

Solids content: 52%
Particle size (LCS): 307 nm

### Viscosity: 105 mPa s

Tg of polyurethaneurea: −78.0° C.

### THERMOPLASTIC FOAM PRODUCED FROM TWO SPECIFIC POLYURETHANE DISPERSIONS (EXAMPLES)

All the foams described here are white and do not include any dyes or pigments.

### PRODUCTION OF THE POLYURETHANE FOAM (COMPARATIVE EXAMPLE 1)

2 kg of the above-described aqueous polyurethane dispersion (A) were mixed with 0.155 kg of a 40% by weight aqueous Pluronic PE6800 solution (from BASF SE, Germany) including 3.45% by weight of citric acid (from Bernd Kraft, Germany) by means of a Dispermat from VMA-Getzmann GmbH. The mixture was then beaten in a Pico-Mix from Hansa-Mixer to give a foam having a density of 200 g/l. The beaten foam was cast onto a polyolefin-coated release paper (Felix Schoeller Y05200) of width 30 cm at a coater setting of 3000 μm. This foam was dried at 120° C. for 30 minutes.

TABLE 1
Foam properties of comparative example 1

| | | |
|---|---|---|
| Parameter: | Unit | Result: |

| Thickness | | mm | 1.1 |
|---|---|---|---|
| Foam density (dry) | | g/l | 222 |
| Crack area | | % | 5.3 |
| Crack width | Average | mm | 2.46 |
| | SD | mm | 1.44 |
| | Smallest value | mm | 0.54 |
| | Greatest value | mm | 7.97 |
| Crack depth | Average | mm | 0.39 |
| | SD | mm | 0.12 |

| | | |
|---|---|---|
| Viability | % | 75 |
| Tensile stress at break | kPa | 1095 |
| Elongation at break | % | 239 |
| Average pore size | μm | 427 |

### PRODUCTION OF THE POLYURETHANE FOAM (COMPARATIVE EXAMPLE 2)

4.5 kg of the above-described aqueous polyurethane dispersion (A) were mixed with 0.349 kg of a 40% by weight aqueous Pluronic PE6800 solution (from BASF SE, Germany) including 3.45% by weight of citric acid (from Bernd Kraft, Germany) and 0.225 kg of Niax L6889 (from Momentive Performance Materials Inc., USA) by means of a Dispermat from VMA-Getzmann GmbH. The mixture was then beaten in a Pico-Mix from Hansa-Mixer to give a foam having a density of 200 g/l. The beaten foam was cast onto a polyolefin-coated release paper (Felix Schoeller Y05200) of width 30 cm at a coater setting of 3000 μm. The foam was dried at 130 to 140° C. for 7 minutes.

TABLE 2
Foam properties of comparative example 2

| | | |
|---|---|---|
| Parameter: | Unit | Result: |

| Thickness | | mm | 2.4 |
|---|---|---|---|
| Foam density (dry) | | g/l | 128 |
| Crack area | | % | — |
| Crack width | Average | mm | — |
| | SD | mm | — |
| | Smallest value | mm | — |
| | Greatest value | mm | — |
| Crack depth | Average | mm | — |
| | SD | mm | — |

| | | |
|---|---|---|
| Viability | % | 18 |
| Tensile stress at break | kPa | 334 |
| Elongation at break | % | 208 |
| Average pore size | μm | 431 |

### PRODUCTION OF THE POLYURETHANE FOAM (INVENTIVE EXAMPLE 1)

3.19 kg of the above-described aqueous polyurethane dispersion (A) were mixed with 1.367 kg of polyurethane dispersion (B), 0.91 kg of a 10% by weight aqueous Rheolate 208 dispersion (from Elementis Specialties, Germany) and 0.353 kg of a 40% by weight aqueous Pluronic PE6800 solution (from BASF SE, Germany) including 3.45% by weight of citric acid (from Bernd Kraft, Germany) by means of a Dispermat from VMA-Getzmann GmbH. The mixture was then beaten in a Pico-Mix from Hansa-Mixer to give a foam having a density of 200 g/l. The beaten foam was cast onto a polyolefin-coated release paper (Felix Schoeller Y05200) of width 30 cm at a coater setting of 3000 μm. The foam was then dried at 130 to 140° C. for 7 minutes.

TABLE 3
Foam properties of inventive example 1

| | | |
|---|---|---|
| Parameter: | Unit | Result: |

| Thickness | | mm | 1.8 |
|---|---|---|---|
| Foam density (dry) | | g/l | 159 |
| Crack area | | % | 0.0 |
| Crack width | Average | mm | — |
| | SD | mm | — |
| | Smallest value | mm | — |
| | Greatest value | mm | — |
| Crack depth | Average | mm | — |
| | SD | mm | — |

| | | |
|---|---|---|
| Viability | % | 85 |
| Tensile stress at break | kPa | 377 |
| Elongation at break | % | 248 |
| Average pore size | μm | 467 |

No detectable cracks were detected in the foam produced according to inventive example 1, which is the reason why there are no figures for crack width and crack depth in table 3. Moreover, on comparison of the results from tables 1 to 3, it is apparent that the inventive mixture of dispersions (A) and (B) gives an article that has been foamed in accordance with the invention and has no cracks and nevertheless has high viability. The foamed articles according to the comparative examples have either a low viability or cracks.

1. A foamed article produced by mixing a first polyurethane dispersion (A) with at least one second polyurethane dispersion (B), optionally with addition of further additives, foaming and subsequently drying the mixture, wherein the polyurethane dispersion (A) is obtained by preparing
A) isocyanate-functional prepolymers from
A1) organic polyisocyanates,
A2) polymeric polyols having number-average molecular weights of 400 to 8000 g/mol and OH functionalities of 1.5 to 6, and
A3) optionally hydroxyl-functional compounds having molecular weights of 62 to 399 g/mol, and
A4) optionally isocyanate-reactive, anionic or potentially anionic and optionally nonionic hydrophilizing agents,
and
B) the free NCO groups thereof are then wholly or partly reacted
B1) optionally with amino-functional compounds having molecular weights of 32 to 400 g/mol and
B2) with amino-functional, anionic or potentially anionic hydrophilizing agents
by chain extension and the prepolymers are dispersed in water before, during or after step B), and
wherein the second polyurethane dispersion (B) is obtained by reactive conversion of at least the following components:
A. an aliphatic polyisocyanate component having an average isocyanate functionality of ≥1.8 and ≤2.6,
B. a polymeric polyether polyol component,
C. an amino-functional chain extender component having at least 2 isocyanate-reactive amino groups, comprising at least one amino-functional compound C1. that does not have any ionic or ionogenic groups and/or an amino-functional compound C2. that has ionic or ionogenic groups,
D. optionally further hydrophilizing components other than C2.,
E. optionally hydroxyl-functional compounds having a molecular weight of 62 to 399 mol/g,
F. optionally further polymeric polyols other than B.,
G. a compound having exactly one isocyanate-reactive group or a compound having more than one isocyanate-reactive group where only one of the isocyanate-reactive groups reacts with the isocyanate groups present in the reaction mixture under the reaction conditions chosen, and
H. optionally an aliphatic polyisocyanate component having an average isocyanate functionality of >2.6 and ≤4,
where components B. and F. together contain ≤30% by weight of component F., based on the total mass of components B. and F.

2. The foamed article as claimed in claim 1, wherein the foamed article has at least one of the following properties i. to xiii.:
i. the foamed article has no cracks having a width of 2 mm;
ii. the foamed article has no surface cracks deeper than 0.4 mm;
iii. the foamed article has a crack area of 6%, based on the total area of the foamed article;
iv. the foamed article has a cell viability of 70% or a classification of 0 to 2 in a cytotoxicity test based on DIN ISO 10993-5:2009-10;
v. the foamed article has a density within a range from 80 to 500 g/l;
vi. the foamed article has a thickness (D) within a range from 0.1 to 100 mm;
vii. the foamed article has a tensile stress at break within a range from 100 to 1100 kPa measured according to DIN EN ISO 527-2:2012-06;
viii. the foamed article has an elongation at break within a range from 100% to 500%, measured according to DIN EN ISO 527-2:2012-06;
ix. the polyurethaneurea formed from the polyurethane dispersion (B) is amorphous and has a Tg ≤−25° C., determined by differential scanning calorimetry in accordance with DIN EN 61006, Method A;
x. the foamed article has a melting or softening range of 180° C., at a maximum pressure of 4 bar and has full thermoplastic processability within this range;
xi. the foamed article has an average pore size within a range from 200 to 750 μm;
xii. the foamed article has an outer layer at least on one surface of the foamed article;
xiii. the foamed article is part of a composite material.

3. The foamed article as claimed in claim 1, wherein the weight ratio of the polyurethane dispersion (A) to the polyurethane dispersion (B) is within a range from 1:1 to 5:1, based on the total mass of the masses of dispersions (A) and (B).

4. The foamed article as claimed in claim 1, wherein a film formed from the dispersion (B) has a tensile strain at break of ≤5 MPa combined with an elongation at break of 1750%.

5. The foamed article as claimed in claim 1, wherein the hydrophilizing agents B2) used are hydrophilizing agents containing sulfone groups.

6. The foamed article as claimed in claim 1, wherein the dispersion (A) has a solids content of polyurethane of 52% to 65% by weight, based on the total mass of the dispersion (A).

7. The foamed article as claimed in claim 1, wherein component A. or A1) is isophorone diisocyanate and/or hexamethylene diisocyanate.

8. The foamed article as claimed in claim 1, wherein component B. comprises or consists of poly(tetramethylene glycol) polyether polyols.

9. The foamed article as claimed in claim 1, wherein component B. comprises or consists of a mixture of at least two poly(tetramethylene glycol) polyether polyols, wherein the at least two poly(tetramethylene glycol) polyether polyols are of different number-average molecular weight.

10. The foamed article as claimed in claim 1, wherein component D. comprises nonionically hydrophilizing components.

11. The foamed article as claimed in claim 1, wherein component H. is used and the molar ratio of component G. to component H. is 5:1 to 1:5.

12. The foamed article as claimed in claim 1, wherein the polyurethane dispersion (B) is obtained by preparing isocyanate-functional polyurethane prepolymers a) from components A., B. and optionally D. and/or C2., and optionally compounds E. and/or H., and the free NCO groups thereof are then wholly or partly reacted with the amino-functional chain-extender component C., and also component G. and optionally components D. and H.

13. A process for producing a foamed article as claimed in claim 1, wherein the process comprises:
(V1) mixing the polyurethane dispersion (A) and the polyurethane dispersion (B) to obtain a mixture (M1),
(V2) optionally adding to (M1) at least one interface-active substance,
(V3) optionally adding to (M1) at least one surfactant,
(V4) optionally adding to (M1) a thickener,
(V5) foaming the mixture (M1) to give a foam,
(V6) optionally maturing the foam from step (V2),
(V7) optionally applying the foam formed in step (V2) or (V3) to a substrate,
(V8) drying the foam to form the foamed article.

14. A wound dressing, a hygiene article, or a wearable patch, comprising the foamed article as claimed in claim 1.

15. A kit of parts at least including a polyurethane dispersion (A) and a polyurethane dispersion (B), wherein the polyurethane dispersion (A) or the polyurethane dispersion (B) includes an added material (C), wherein the dispersion (A) or (B) including the added material (C) has a cell viability of 70% in a cytotoxicity test or a classification of 0 to 2 according to DIN ISO 10993-5:2009-10.
